(19) United States
(12) Patent Application Publication
(10) Pub. No.: US 2023/0355818 A1
(43) Pub. Date: Nov. 9, 2023

(54) SYSTEM FOR REDUCING MICROBIAL BURDEN ON A SURFACE

(71) Applicant: STERIFRE MEDICAL, INC., Kirkland, WA (US)

(72) Inventors: Mark Golkowski, Kirkland, WA (US); Czeslaw Golkowski, Kirkland, WA (US)

(21) Appl. No.: 17/926,541

(22) PCT Filed: May 19, 2021
(86) PCT No.: PCT/US2021/033233

§ 371 Date: Nov. 18, 2022

Related U.S. Application Data
(60) Provisional application No. 63/027,170, filed on May 19, 2020.

(51) Int. Cl. A61L 2/24; A61L 2/20

(52) U.S. Cl. CPC A61L 2/24; A61L 2/202; A61L 2/208; A61L 2101/02

(57) ABSTRACT

A method for reducing viable microbial burden on a surface. The method includes placing an item into a system chamber. The method includes a conditioning phase where ozone is generated by an ozone generator and a fan circulates the ozone in a closed loop between the ozone generator and the system chamber. The method then includes a disinfection phase where a pump pumps disinfectant to a nebulizer where it is converted into a disinfectant vapor. A fan is then activated to circulate the vapor in a closed loop between the nebulizer and the system chamber. After the disinfecting phase, the method activates a post-disinfection conditioning phase where an ozone generator generates ozone that is circulated by a fan in a closed loop between the ozone generator, the nebulizer and the system chamber. Lastly, the method activates a system clearing phase, where air flow is pulled into the system through an inlet and exhausted out of the system through an outlet.

### BACKGROUND

### Technical Field

Several embodiments of the present disclosure relate generally to the art of generating atmospheres having sterilizing, disinfecting, sanitizing, decontaminating, and/or therapeutic aspects, and more particularly to sterilization, disinfection, sanitization, and/or decontamination of therapeutic devices, as well as related systems and methods.

### Description of the Related Art

Sterilization, disinfection, sanitization, and decontamination methods are used in a broad range of applications. A variety of methods is used, including steam, chemicals, fumigants, radiation, among others. Drawbacks to these methods exist and are addressed by the systems and methods disclosed herein.

### SUMMARY

As disclosed herein, a variety of items or surfaces may require processing in order to reduce the bioburden and decrease risk of infections. For example, critical items (such as surgical instruments, which contact sterile tissue), semi-critical items (such as endoscopes, which contact mucous membranes), and noncritical items (such as stethoscopes, which contact only intact skin) require various types of treatment, for example sterilization, high-level disinfection, and low-level disinfection, respectively. The present disclosure provides for various systems and methods for disinfecting/sanitizing various items (e.g., medical devices or electronics) and surfaces (e.g., workspaces, patient rooms, organic material, including but not limited to patient wounds).

Various systems and methods are provided for herein in order to accomplish disinfection of one or more items, surfaces etc. Additionally, in several embodiments the systems and methods are configured to allow low or high level disinfection. In still additional embodiments, the systems and methods are configured to allow sterilization.

For example, provided for herein in several embodiments is a system for reducing the viability of microorganisms on a surface, comprising a chamber, a cartridge, a reservoir, a nebulizer, at least one pump, an ozone generator, a fan, an inlet, and outlet, and a plurality of valves. In some embodiments, the chamber is configured to receive an item to be disinfected, sterilized, or sanitized. In some embodiments, the cartridge is configured to contain a solution comprising hydrogen peroxide. In some embodiments, the reservoir is configured to receive excess hydrogen peroxide solution. In some embodiments, the nebulizer is configured to convert hydrogen peroxide into a vapor. In some embodiments, the at least one pump is configured to deliver hydrogen peroxide to the nebulizer. In some embodiments, the at least one pump includes a first peristaltic pump and a second peristaltic pump. In some embodiments, the first peristaltic pump is fluidly connected to the cartridge and the reservoir. In some embodiments, the second peristaltic pump is fluidly connected to the reservoir and the nebulizer, wherein the second peristaltic pump is configured to deliver hydrogen peroxide from the reservoir to the nebulizer. In some embodiments, the ozone generator is configured to generate ozone. In some embodiments, the fan is configured to circulate air, including ozone, through the system. In some embodiments, the inlet is configured to allow air to flow into the system. In some embodiments, the outlet is configured to allow air to flow out of the system. In some embodiments, the first valve is configured to control fluid flow into and out of the system. In some embodiments, the second valve is configured to control fluid flow to the nebulizer.

In some embodiments, the system does not include a heater. In some embodiments, the system does not include a humidifier or a dehumidifier. In some embodiments, the system does not include a desiccator. In some embodiments, the blower maintains the system at a slight negative pressure. In some embodiments, the system further includes a fluid flow sensor intended to maintain constant fluid flow in the system. In some embodiments, the system is configured to operate at a temperature between 20° C. to 25° C. In some embodiments, the system is configured to operate with a relative humidity between 20% and 60%. In some embodiments, the second peristaltic pump is configured to deliver a predetermined quantity of hydrogen peroxide solution to the nebulizer. In some embodiments, the first peristaltic pump is configured to maintain a predetermined quantity of hydrogen peroxide solution in the reservoir.

In some embodiments, the above disclosed system can be used in a method for reducing viable microbial burden. For example, provided herein in several embodiments is a method for reducing viable microbial burden on a surface comprising placing at least one item into a chamber of a system presently disclosed. In some embodiments, the method includes activating a conditioning phase to circulate ozone in the system. In some embodiments, the method includes activating a disinfection phase wherein the hydrogen peroxide solution is nebulized and is circulated through the system. In some embodiments, the method includes activating a post-disinfection conditioning phase to circulate ozone in the system. In some embodiments, the method includes activating a system clearing phase to pull air into the system through the inlet and exhaust the air out of the outlet.

In some embodiments, a method for reducing viable microbial burden on a surface is disclosed, the method comprising placing at least one item into a chamber of a system for reducing microorganism viability, wherein the system comprises a nebulizer configured to convert hydrogen peroxide solution into a vapor, a cartridge configured to contain the hydrogen peroxide solution, at least one peristaltic pump, an ozone generator, a blower, an inlet and an outlet. In some embodiments, the method includes activating a conditioning phase to circulate ozone from the ozone generator in the system, wherein the ozone is configured to convert H₂O molecules to OH radicals so as to reduce residual moisture in the system. In some embodiments, the method includes activating a disinfection phase wherein the hydrogen peroxide solution is nebulized into a spray and is circulated through the system. In some embodiments, the method includes activating a post-disinfection conditioning phase to circulate ozone from the ozone generator in the system, wherein the ozone is configured to neutralize any remaining H₂O₂ in the system. In some embodiments, the method includes activating a system clearing phase to pull air into the system through the inlet, circulate the air through the nebulizer and the chamber, and exhaust the air out of the outlet.

In some embodiments, the method includes a disinfection phase that operates at a temperature between about 20° C. to 25° C. In some embodiments, the system of the disclosed method operates with a relative humidity between about 20% and 60%. In some embodiments, the method includes conditioning phase that with a duration of at least 2.5 minutes. In some embodiments, the method includes a disinfection phase with a duration of at least 4.5 minutes. In some embodiments, the method includes a post-disinfection phase with a duration of at least 2 minutes. In some embodiments, the method includes a system clearing phase with a duration of at least 1 minute. In some embodiments, the system of the method does not include a heater configured to dry the system. In some embodiments, the system of the disclosed method does not include a humidifier or a dehumidifier. In some embodiments, the system of the disclosed method does not include a desiccator. In some embodiments, the fluid flow during the conditioning phase of the disclosed method circulates fluid flow that bypasses the nebulizer. In some embodiments, fluid flow during the disinfection phase of the disclosed method circulates fluid flow through the nebulizer. In some embodiments, fluid flow during the post-disinfection conditioning phase of the disclosed method circulates fluid flow through the nebulizer. In some embodiments, the fluid flow during the clearing phase of the disclosed method circulates fluid flow that bypasses the nebulizer.

In some embodiments, disclosed is a system for reducing the viability of microorganisms on a surface comprising a chamber, a reservoir, a peristaltic pump, an ozone generator, a nebulizer, a fan, an inlet and an outlet. In some embodiments, the chamber is configured to contain an item to be sterilized, disinfected, sanitized, or decontaminated. In some embodiments, the reservoir is configured to contain a disinfectant. In some embodiments, the peristaltic pump is connected to the reservoir. In some embodiments, the ozone generator is configured to generate ozone. In some embodiments, the nebulizer is configured to convert disinfectant into a vapor, wherein the peristaltic pump is configured to deliver disinfectant from the reservoir to the nebulizer. In some embodiments, the fan is configured to circulate air, including ozone, through the system and chamber. In some embodiments, the inlet is configured to allow air to flow into the system. In some embodiments, the outlet is configured to allow air to flow out of the system.

In some embodiments, the system includes an inlet that is fluidically connected to the ozone generator. In some embodiments, the system includes a valve that is configured to be opened or closed to allow or prevent air flow from the inlet to the ozone generator. In some embodiments, the system includes a valve that is configured to control fluid flow between the fan and the ozone generator. In some embodiments, the system includes a valve that is configured to close such that fluid flow from the fan is blown through the outlet. In some embodiments, the system includes a disinfectant concentration that is between about 30% to 60%. In some embodiments, the system includes a disinfectant concentration that is about 50%. In some embodiments, the system includes a disinfectant is hydrogen peroxide. In some embodiments, the hydrogen peroxide concentration of the system is about 50%. In some embodiments, the system has a reservoir that includes a replaceable cartridge. In some embodiments, the system is configured to operate at a temperature between about 20° C. to 25° C. In some embodiments, the system includes a peristaltic pump that is configured to provide a flow rate of less than about 1 ml/min of hydrogen peroxide. In some embodiments, the system is configured to operate with a relative humidity between about 20% and 60%. In some embodiments, the system includes an inlet with a high efficiency particulate air (HEPA) filter. In some embodiments, the system includes an outlet with an activated carbon filter or a high efficiency particulate air (HEPA) filter. In some embodiments, the system includes a sensor disposed in the chamber and configured to sense a level of at least one of humidity, pressure, and temperature within the chamber. In some embodiments, the system includes a peristaltic pump that is configured to receive the cartridge. In some embodiments, the system includes a peristaltic pump configured to deliver a predetermined quantity of hydrogen peroxide solution to the nebulizer. In some embodiments, the system includes a cartridge that includes the disinfectant.

In some embodiments, disclosed is a method for reducing viable microbial burden on a surface. In some embodiments the method includes placing at least one item into a chamber configured to contain the at least one item. In some embodiments, the method includes activating a conditioning phase. In some embodiments, the conditioning phase can include activating a fan to circulate air in a closed loop to circulate the chamber, activating an ozone generator to generate ozone, activating the fan to circulate air, including the ozone, in the closed loop between the ozone generator and the chamber. In some embodiments, the method can include activating a disinfection phase. In some embodiments, the disinfection phase can include pumping disinfectant with a peristaltic pump from a reservoir to a nebulizer, converting disinfectant into a vapor with the nebulizer, activating the fan to circulate air, including the vapor, in the closed loop between the nebulizer and the chamber, and activating the fan to circulate air, including the ozone, in the closed loop between the ozone generator and the chamber. In some embodiments, the method includes activating a post-disinfection conditioning phase. In some embodiments, the post-disinfection conditioning phase includes activating an ozone generator to generate ozone and activating the fan to circulate air, including the ozone, in the closed loop between the ozone generator, the nebulizer, and the chamber. In some embodiments, the method includes activating a system clearing phase. In some embodiments, the system clearing phase includes activating a valve to allow air to flow into the system through an inlet, activating a valve to allow air to flow out of the system through an outlet, and activating the fan to introduce the air through the inlet, into the chamber, and exhaust through the outlet.

In some embodiments, the method is performed in about 10 minutes. In some embodiments, the conditioning phase of the method is about 150 seconds in duration. In some embodiments, the post-disinfection conditioning phase of the method is about 2 minutes in duration. In some embodiments, the sterilization or disinfection phase of the method is about 4 minutes and 30 seconds to about 5 minutes in duration. In some embodiments, the system clearing phase of the method is about 60 seconds. In some embodiments, the system of the method includes an inlet that comprises a HEPA filter. In some embodiments the system clearing phase of the method further comprises closing a valve to allow the fan to push air through the outlet. In some embodiments, the system of the method includes an outlet that comprises an activated carbon filter and a high efficiency particulate air (HEPA) filter. In some embodiments, the method includes disinfectant at a concentration of between about 30% to 60%. In some embodiments, the method includes disinfectant at a concentration of about 50%. In some embodiments, the method includes disinfectant that is hydrogen peroxide. In some embodiments, the method includes hydrogen peroxide at a concentration of about 50%. In some embodiments, the method includes a reservoir with a replaceable cartridge. In some embodiments, the method is operated at a temperature between about 20° C. to 25° C. In some embodiments, the method is operated at a relative humidity between about 20% and 60%. In some embodiments, the method is operated at or below an ambient pressure.

In some embodiments, disclosed is an automated method for sterilizing or disinfecting at least one item. In some embodiments, the method includes receiving at least one item to be sterilized or disinfected into an interior volume of a chamber for sterilization or disinfection. In some embodiments, the chamber for sterilization or disinfection is part of a system comprising an inlet, an outlet port, an ozone generator, a sterilant generator, and a plurality of conduits configured to fluidly connect each of the inlet, sterilant generator, ozone generator, and the chamber. In some embodiments, the system includes at least one fan, configured to provide gaseous flow through the system. In some embodiments, the system includes a controller and a plurality of valves in respective conduits. In some embodiments, the method includes activating a conditioning phase by the controller, wherein the conditioning phase is configured to dry a surface of the at least one item in the chamber and internal flow conduits, wherein the controller activates the fan to move air, and wherein the valves are positioned by the controller to provide closed loop flow of air moved by the fan. In some embodiments, the method includes activating an disinfection phase by the controller, wherein the exposure phase is configured to disinfect the at least one item, wherein the controller causes the disinfectant generator to begin generating disinfectant, wherein the disinfectant comprises a mist of hydrogen peroxide generated from a solution of hydrogen peroxide in the disinfectant generator at a concentration of about 50%, wherein the valves are positioned by the controller to provide closed loop flow through the nebulizer so that disinfectant is delivered to the chamber for a pre-determined time to disinfect the at least one item. In some embodiments, the method includes activating a post-disinfection conditioning phase by the controller, wherein the post-disinfection phase introduces ozone generated by the ozone generator into the chamber containing residual hydrogen peroxide disinfectant to neutralize the disinfectant. In some embodiments, the method includes activating a system clearing phase by the controller, wherein the purge phase includes positioning the valves by the controller to allow open flow and to allow air to be pulled in through the inlet and force the gaseous water vapor and oxygen from the chamber and out the outlet, wherein each of the inlet and outlet comprise a respective filter.

In some embodiments, the automated method includes a controller that activates the fan to move air through the ozone generator to produce ozone. In some embodiments, the automated method includes a disinfectant comprising a vapor of hydrogen peroxide. In some embodiments, the automated method operates at a preprogrammed relative humidity between about 20% to 60%. In some embodiments, the conditioning phase of the automated method is activated for about 180 seconds. In some embodiments, the disinfection phase of the automated method is activated for about 4 minutes and 30 seconds. In some embodiments, the post-disinfection conditioning phase of the automated method is activated for about 120 seconds. In some embodiments, the system clearing phase of the automated method is activated for about 60 seconds. In some embodiments, the system of the automated method is configured to receive a cartridge. In some embodiments, the automated method operates between an ambient temperature between about 20° C. to 25° C. In some embodiments, the sterilant of the automated method is delivered by a peristaltic pump. In some embodiments, in the automated method, at least one of the filters of the inlet and outlet is a HEPA filter. In some embodiments, in the automated method, at least one of the filters of the inlet and outlet is a charcoal filter.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1A illustrates a non-limiting embodiment of a system for reducing microorganisms on a surface.

FIG. 1B illustrates a front view of the embodiment of the system for microorganisms on a surface of FIG. 1A, wherein a chamber and cartridge door of the device are opened.

FIG. 2A illustrates a side view of an embodiment of a cartridge for use in an embodiment of the system for reducing microorganisms on a surface; FIG. 2A illustrates the cartridge with an attached lid.

FIG. 2B illustrates a perspective view of the cartridge of FIG. 2A with the lid removed.

FIGS. 2C-2E illustrate a top perspective view, a front view, and a side view of another embodiment of a cartridge for use in an embodiment of the system for reducing microorganisms on a surface.

FIG. 2F illustrates a cross-sectional view of the embodiment of the cartridge as illustrated in FIGS. 2A-2E along Section A-A wherein the spout of the cartridge is closed.

FIG. 2G illustrates a cross-sectional view of the embodiment of the cartridge as illustrated in FIGS. 2A-2E along the Section A-A wherein the spout of the cartridge is opened.

FIGS. 2H-2I illustrate a cross-sectional view of the spout of an embodiment of a cartridge when the spout moves between opened and closed positions.

FIGS. 2J-2K illustrate perspective and a transparent side-view of the tamper-resistant features of an embodiment of a cartridge.

FIGS. 3A-3B illustrate two top perspective views of an embodiment of a cartridge engagement mechanism for use in an embodiment of the system for reducing microorganisms on a surface.

FIGS. 3C-3D illustrate a top and bottom view of the cartridge engagement mechanism of FIGS. 3A-3B.

FIGS. 3E-3F illustrate two top perspective views of the cartridge engagement mechanism configured to receive a top portion of a cartridge for use in an embodiment of the system for reducing microorganisms on a surface.

FIG. 3G illustrates a front view of another embodiment of a cartridge engagement mechanism wherein the cartridge engagement mechanism is in a first position.

FIG. 3H illustrates a front view of the cartridge engagement mechanism of FIG. 3G wherein the cartridge engagement mechanism is in a second position.

FIG. 3I illustrates a side view of the cartridge engagement mechanism of FIGS. 3G-3H wherein the cartridge engagement mechanism is in a first position.

FIG. 3J illustrates a side view of the cartridge engagement mechanism of FIGS. 3G-3I wherein the cartridge engagement mechanism is in a second position.

FIG. 4A illustrates a cross-sectional view of the cartridge engagement mechanism of FIGS. 3G-3I engaged with a cartridge.

FIG. 4B illustrates a cross-sectional view of fluid flow out of the cartridge upon engagement of the cartridge with the cartridge engagement mechanism as shown in FIG. 4A.

FIG. 5A illustrates a front view of an embodiment of an evaporator for use in an embodiment of the system for reducing microorganisms on a surface.

FIG. 5B illustrates a diagram of an embodiment of a combined nebulizer and evaporator for use in an embodiment of the system for reducing microorganisms on a surface

FIG. 6 illustrates a schematic of an embodiment of a system for reducing microorganisms on a surface.

FIG. 7 illustrates a flow chart of an embodiment of a method for reducing microorganisms on a surface

FIG. 8A illustrates an embodiment of the schematic of FIG. 6 during a first Phase of the flow chart of FIG. 7, wherein a chamber of the system for reducing microorganisms on a surface is being conditioned.

FIG. 8B illustrates an embodiment of the schematic of FIG. 6 during a second Phase of the flow chart of FIG. 7, wherein an item placed in the chamber of the system for reducing microorganisms on a surface is being disinfect, sterilized, or sanitized.

FIG. 8C illustrates an embodiment of the schematic of FIG. 6 during a third Phase of the flow chart of FIG. 7, wherein the chamber of the system for sterilization, disinfection, and sanitization is conditioned after the item placed in the chamber has been disinfected, sterilized, or sanitized.

FIG. 8D illustrates an embodiment of the schematic of FIG. 6 during a fourth Phase of the flow chart of FIG. 7, wherein the system is cleared.

FIG. 9 illustrates a schematic of another embodiment of a system for reducing microorganisms on a surface.

FIG. 10 illustrates a flow chart of an embodiment of a method for reducing microorganisms on a surface.

FIG. 11A illustrates an embodiment of the schematic of FIG. 9 during a first Phase of the flow chart of FIG. 10, wherein a chamber of the system for reducing microorganisms on a surface is being conditioned.

FIG. 11B illustrate an embodiment of the schematic of FIG. 9 during a second Phase of the flow chart of FIG. 10, wherein an item placed in the chamber of the system for reducing microorganisms on a surface is being disinfect, sterilized, or sanitized.

FIG. 11C illustrates an embodiment of the schematic of FIG. 9 during a third Phase of the flow chart of FIG. 10, wherein the chamber of the system for sterilization, disinfection, and sanitization is conditioned after the item placed in the chamber has been disinfected, sterilized, or sanitized.

FIG. 11D illustrates an embodiment of the schematic of FIG. 9 during a fourth Phase of the flow chart of FIG. 10, wherein the system is cleared.

FIGS. 12, 13A-13B illustrate an embodiment of a system for reducing microorganisms on a surface.

FIG. 14 illustrates an embodiment of a display screen on the device of FIGS. 12, 13A-13B when the device is ready to receive an item for reducing microorganisms on a surface.

FIG. 15 illustrates an enlarged view of the chamber and a plurality of adjustable shelves of the device of FIGS. 12, 13A-13B wherein a plurality of items are placed on the adjustable shelves for reducing microorganisms on a surface.

FIG. 16 illustrates an enlarged view of the door of the device of FIGS. 12, 13A-13B wherein the items for reducing microorganisms on a surface are viewable through the viewing window of the chamber door.

FIG. 17 illustrates the display screen of FIG. 14 wherein the “start” button is engaged to being the process of reducing microorganisms on a surface.

FIG. 18 illustrates the device of FIGS. 12, 13A-13B during disinfection of the item inserted into the device.

FIG. 19 illustrates the device of FIGS. 12, 13A-13B after disinfection of the item inserted into the device.

FIGS. 20A-20B illustrate the device of FIGS. 12, 13A-13B mounted on a wall or placed on a counter-top.

### DETAILED DESCRIPTION

### General

Sterilization, disinfection, sanitization, and decontamination methods are used in a broad range of applications, and have used an equally broad range of sterilization, disinfection, sanitization, and decontamination agents. The term “sterilization” generally refers to the inactivation of bio-contamination, especially on inanimate objects. The term “disinfection” generally refers to the inactivation of organisms considered pathogenic. Although the term “sterilization” may be used in describing certain embodiments herein, it would be appreciated that, unless otherwise indicated, such embodiments can also be used for disinfection (e.g., high-level disinfection, low-level disinfection, etc.), sanitization, and/or other types of decontamination, e.g., as provided with their regulatory definitions.

Pulsed or silent electric discharge in air or other gases produces non-thermal plasma. Non-thermal plasma processing involves producing plasma in which the majority of the electrical energy goes into the excitation of electrons. These plasmas are characterized by electrons with kinetic energies much higher than those of the ions or molecules. The electrons in these plasmas are short-lived under atmospheric pressure; instead, they undergo collisions with the preponderant gas molecules. The electron impact on gas molecules causes dissociation and ionization of these molecules, which creates a mix of reactive species, in the form of free radicals, reactive oxygen and nitrogen species, ions, and secondary electrons. These reactive species cause unique and diverse chemical reactions to occur, even at relatively low temperatures. These chemical reactions are utilized in low temperature decontamination and sterilization technologies. While there are certain non-thermal plasma devices for wound treatment (or disinfection, sterilization, etc. of devices and objects), prior to the embodiments disclosed herein, all suffered from various therapeutic and practical limitations. First, all of these devices require interaction between the plasma and the wound (or object); that is, since the electric discharge takes place directly on the tissue, the treated tissue itself plays the role of an electrode. This makes the application of non-thermal plasma exquisitely sensitive to small movements or changes in geometry. This adds significant complexity to the treatment and requires the provider to have specialized training to maintain the proper tolerances. Other limitations include the inability to cover large surface areas in a short period of time and equipment that has a large environmental footprint and requires a high upfront cost. Additionally, current commercialized non-thermal plasma devices have a requirement for significant provider training and time to administer treatment including one on one provider to patient care.

As discussed in detail herein, vaporized hydrogen peroxide (VHP) can be used for sterilization. Certain methods of sterilization with VHP include open loop systems, in which the VHP is applied to the items to be sterilized and then exhausted, and closed loop systems, where sterilizing vapors are recirculated.

In closed loop systems, a carrier gas, such as air, is dried and heated prior to flowing past a vaporizer. A hydrogen peroxide aqueous solution is introduced into the vaporizer and which enables this solution to be vaporized. The resulting vapor is then combined with the carrier gas and introduced into a sterilization chamber of varying size, shape, and material. A blower exhausts the carrier gas from the sterilization chamber and recirculates the carrier gas to the vaporizer where additional VHP is added. Between the sterilization chamber and the vaporizer, the recirculating carrier gas passes through a catalytic destroyer (where any remaining VHP is eliminated from the carrier gas), a dryer, a filter and a heater.

U.S. Pat. Application Publication No.: US 2005/0129571 Al by Centanni discloses a closed loop sterilization system. The purpose of using the closed loop is to prevent decrease of the free radical concentration in the circulating effluent. Centanni teaches that there should be a VHP (vapor hydrogen peroxide) destroyer employed in the loop. Centanni teaches that the ozone is mixed with the hydrogen peroxide vapor or microdroplets and the vapor or microdroplets are produced by injecting hydrogen peroxide water solution on a hot plate and thus evaporating it.

As discussed in greater detail herein the present application provides for various systems and related methods for sterilizing, disinfecting, sanitizing, and/or decontaminating a variety of items, ranging from surgical equipment or other medical devices to electronic equipment, as well as services, rooms, and other items including, but not limited to soft goods, foods, and related manufacturing equipment. A general overview will be provided, with additional detail related to each of the components of such systems provided below. As mentioned above, the term “sterilization” shall be appreciated to not only encompass the removal of all or substantially all microorganisms and or other pathogens from an object or surface but shall also encompass (unless otherwise specified) disinfection, sanitizing, and decontamination.

### Overview

The present application discusses concepts relating to removing and/or reducing the presence of viable microorganisms on a surface. This discussion is intended to cover concepts of sterilization, disinfection, sanitization, and decontamination. Decontamination is generally defined as killing some bacteria and fungi while deactivating viruses. Disinfection and sanitization are two levels of decontamination; “disinfection” refers to killing nearly 100% of germs on surfaces or objects while “sanitization” refers to lowering the number of microorganisms to a safe level by either cleaning or disinfecting. Sterilization, on the other hand, refers to the killing of all microorganisms, viruses, and bacterial spores. Each of these concepts refer to a different level of removing and/or reducing the viability of microorganisms on a surface. Unless specified otherwise, reference to a system or method for removing and/or reducing the presence of microorganisms on a surface is intended to encompass all level of reducing microbial burden/viability (e.g., disinfection, sanitization, decontamination, and sterilization).

The prevention of acquired infections, whether in a commercial, home, or healthcare setting, is an important concern. This can be particularly difficult during a viral outbreak when frequently used items must be regularly disinfect, sterilized, and/or sanitized to prevent spread the spread of the virus. Existing methods of disinfection, sterilization, and/or sanitization are inadequate or burdensome. For example, disinfectant wipes can be ineffective if contact time is insufficiently long or proper protocols are not followed. As well, seams and irregular surfaces can be difficult to reach using manual methods of disinfection, sterilization, and or sanitization. UV systems may be capital intensive, may not be EPA or FDA registered, and may have difficulty treating resilient organisms such as spores. Harsh chemical can damage devices and the exposure of individuals to chemical disinfectants can cause health risks. Lastly, the use of disposable methods of disinfection, sterilization, and/or sanitization can cause a significant environmental impact.

Disclosed are systems and methods for reducing microorganisms on a surface. As will be discussed in more detail below, the system for reducing microorganisms on a surface can be a fully automated system that is intended to disinfect hard non-porous surfaces for reusable non-critical medical devices and general-use items used in healthcare facilities. The disclosed systems and methods provide for rapid and effective broad spectrum disinfection of items used in various settings (e.g., patient care settings) that offer consistent disinfection for patients, healthcare workers, and equipment used in those settings. Although discussions of the use of the disclosed systems and methods may be focused predominantly on healthcare settings, the disclosed systems and methods can be intended for home, commercial, or field use.

In some embodiments, the disclosed system is configured to operate at ambient temperature and ambient pressure conditions in a continuous closed loop flow throughout the disinfection, sterilization, and/or sanitization process.

The system for reducing the viability of microorganisms on a surface includes a chamber for receiving the items for reducing the viability of microorganisms on a surface. In some examples, the system can include a chamber with a plurality of removable shelves on which items for disinfection can be placed.

The system for reducing the viability of microorganisms on a surface can include a 50% hydrogen peroxide as the active ingredient for reducing the viability of microorganisms on a surface. In some embodiments, the 50% hydrogen peroxide is packaged in cartridges that can be removed and replaced from the system when the hydrogen peroxide solution is consumed. In some examples, the hydrogen peroxide can be introduced into the system for reducing microorganisms on a surface using a nebulizer that is configured to convert the hydrogen peroxide solution into a micro-spray that inactivates the microorganisms. In some examples, the system can include an ozone generator that produces ozone to condition the system chamber prior to and after the disinfection, sterilization, and/or sanitization process.

The system for reducing the viability of microorganisms on a surface can be configured such that once the disinfection, sterilization, and/or sanitization is completed, fresh air is automatically introduced into the system through a HEPA filter to flush out the system chamber before the disinfected, sterilized, and/or sanitized items are removed. After the disinfection, sterilization, and/or sanitization process is completed, the air that exits the system chamber can be exhausted through a HEPA and an activated carbon filter to ensure substantially only or only clean air leaves the system.

In some embodiments, the system for reducing the viability of microorganisms on a surface can be a fully integrated system that includes hardware, electronics, and software to operate and monitor the system. The system can be programmed to automatically disinfect, sterilize, and/or sanitize the items placed in the device with the push of a button by the user.

### Systems for Disinfection, Sanitization, and/or Sterilization

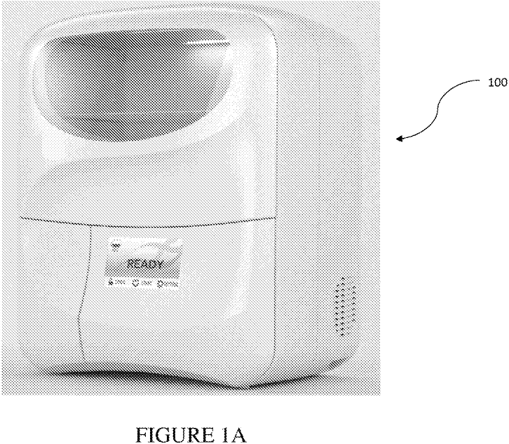

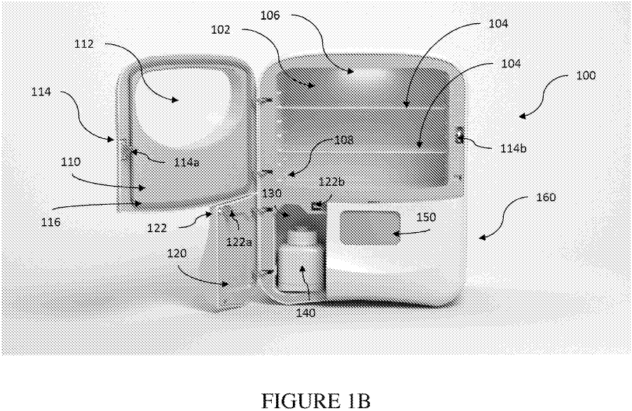

FIGS. 1A-1B illustrate an embodiment of the system for reducing the viability of microorganisms on a surface 100. The system 100 can be configured to be fully automated in order to disinfect hard non-porous surface of reusable non-critical medical devices and general-use items found in healthcare facilities.

In some embodiments, the system for reducing the viability of microorganisms on a surface 100 can include a housing 160 that contains a chamber 102 for receiving an item for reducing microorganisms on a surface. The housing 160 can be configured to house a cartridge 200, a nebulizer (not shown), an evaporator (not shown), an ozone generator (not shown), a plurality of pumps, a plurality of filters, a plurality of valves, and at least one circulating fan.

In some examples, the system for reducing the viability of microorganisms on a surface 100 can have a width of about 40 to about 50 cm (e.g., 44.2 cm), a height of about 40 to 50 cm (e.g., 47.0 cm), and a depth of about 30 to about 40 cm (e.g., 34.1 cm). In some embodiments, the weight of the system 100 can be 25 lbs. In some examples, the system 100 can require an input voltage of 120 V. In some embodiments, the system 100 has a power consumption less than 100 Watts.

### Housing

FIGS. 1A-1B illustrate an embodiment of the housing 160 of the system for reducing microorganisms on a surface 100. In some embodiments, the housing 160 can include a chamber 102 for receiving an item for reducing microorganisms on a surface, a chamber 130 for receiving a cartridge 140, and a screen 150 configured to provide a user interface with the patient.

As show in FIG. 1B, the chamber 102 can be configured to receive one or more items for reducing microorganisms on a surface. In some embodiments, the chamber 102 can have a volume of 14 liters. In some embodiments, the inner dimensions of the chamber 102 can have a width of about 25 to 25 cm (e.g., 31 cm), a height of about 20 to 30 cm (e.g., 23 cm), and a depth of about 15 to 25 cm (e.g., 20 cm). The chamber 102 can include a plurality of pegs (or other protrusions) 108 that are configured to receive and/or engage with at least one adjustable shelf 104. In some embodiments, the adjustable shelves 104 can allow more than one item to be disinfected, sterilized, and/or sanitized at the same time. In some embodiments, this can be done by ensuring that the items to be disinfected, sterilized, and/or sanitized are not in contact with each other, does not overlap itself, and does not touch any of the chamber walls of the system 100. In some examples, the adjustable shelves 104 can be a wire frame with large openings to allow for the circulation of disinfectant/sterilant throughout the chamber 102. The adjustable shelves 104 can provide support for the items to be disinfected, sterilized, and/or sanitized with minimal contact. This can ensure that as much of the surface of the item as possible is in contact with the disinfectant/sterilant.

In some examples, the housing 160 includes a chamber door 110 that seals the chamber 102 and prevents disinfectant/sterilant from escaping the chamber 102 when the item is being disinfected, sterilized, and/or sanitized. In some embodiments, the chamber door 110 can include an engagement portion 114 that secures the chamber door 110 to the body of the housing 160 around the chamber 102. The engagement portion 114 can include a male portion 114a and a female portion 114b. As will be discussed in more detail below, the engagement portion 114 can allow the chamber 102 to be opened only when all disinfectants/sterilants have been neutralized. The chamber door 110 can include a seal 116 that is disposed about an inner surface of the chamber 102 and is configured to engage with the opening of the chamber 102 to ensure that the chamber 102 is sealed when the chamber door 110 is closed such that no disinfectant/sterilant is allowed to escape the chamber 102 when the item is being disinfected, sterilized, and/or sanitized. In some examples, the seal 116 can be made of rubber or any kind of similar material that would allow the chamber 102 to be sealed.

In some embodiments, the chamber door 110 includes a viewing window 112. The viewing window 112 can allow the user to see the items being sterilized. In some examples, the chamber 102 can include a light 106 located in the surface of wall of the chamber 102. The light 106 can be configured to illuminate the interior of the chamber 102 in a variety of colors that is visible by the user through the viewing window 112. In some embodiments, the light 106 can change to indicate where in the disinfecting, sterilizing, or sanitizing cycle the system 100 currently is in.

The system for reducing the viability of microorganisms on a surface 100 can include a chamber 130 that is configured to receive a cartridge 140. As illustrated in FIG. 1B, the chamber 130 includes a cartridge door 120 that houses and/or secures the cartridge 140 in the housing 160 of the system 100. As will be discussed in more detail below, the cartridge 140 contains the disinfectant/sterilant that is used by the system 100. The location of the cartridge door 120 allows the user to easily access the chamber 130 and replace and remove the cartridge 140 when the disinfectant/sterilant stored in the cartridge 140 is consumed. In some embodiments, the cartridge door 120 can include an engagement portion 122 that secures the cartridge door 120 to the body of the housing 160 around the chamber 130. The engagement portion 122 can include a male portion 122a and a female portion 122b.

In some embodiments, the screen 150 is located on an outside surface of the housing 160. The screen 150 can be configured to provide information to the user on the status of the disinfection, sterilization, and sanitization of the items in the chamber 102. The screen 150 can also provide a user interface that allows the user to select disinfecting, sterilizing, and/or sanitizing of the items put in the chamber 102.

### Cartridge

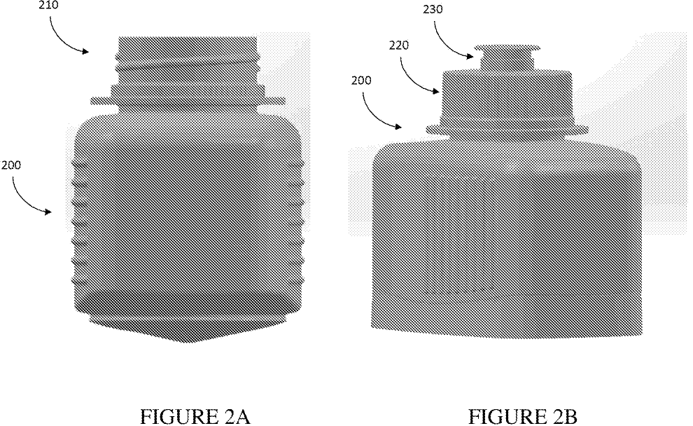

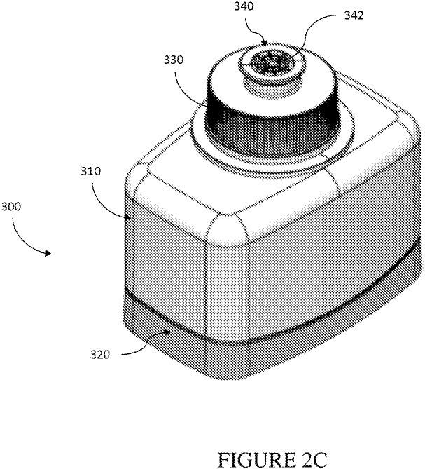

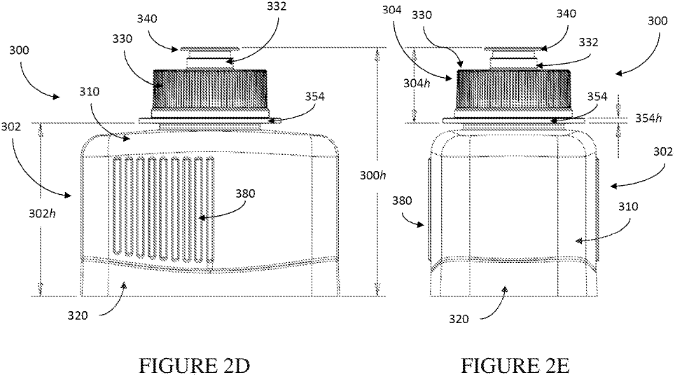

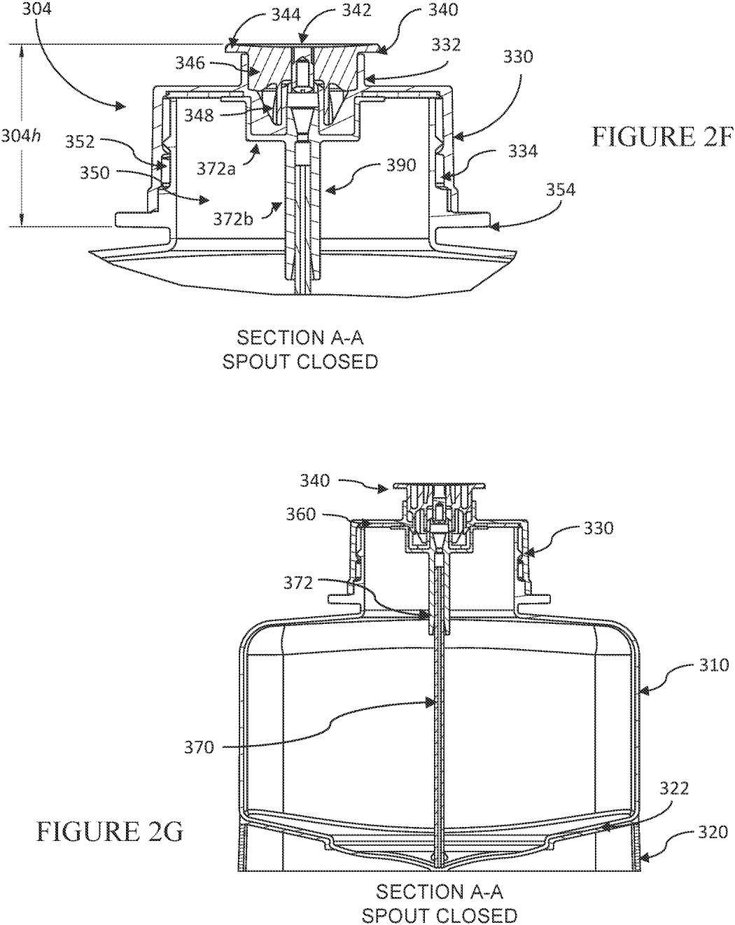

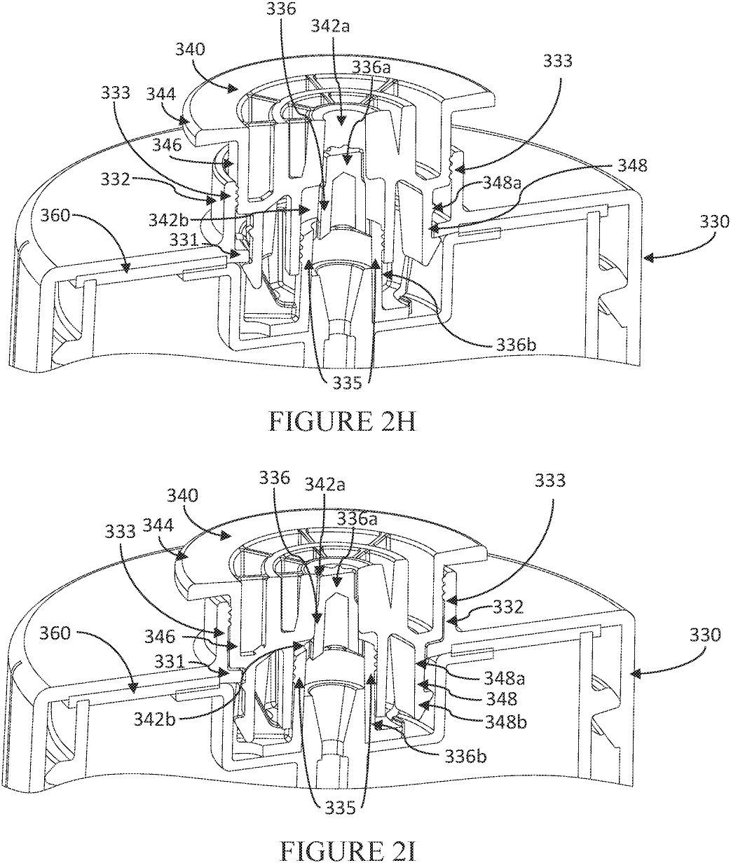

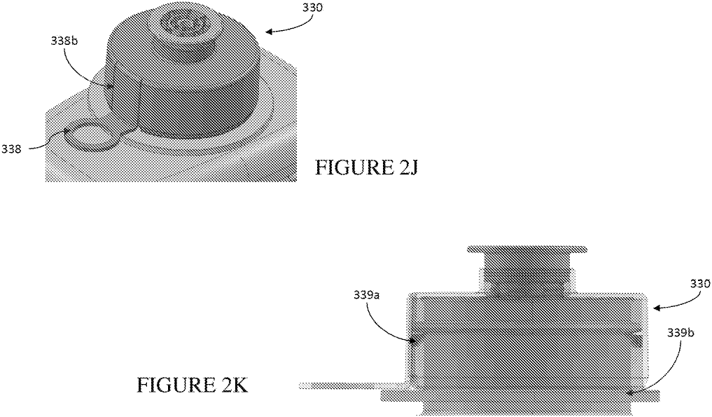

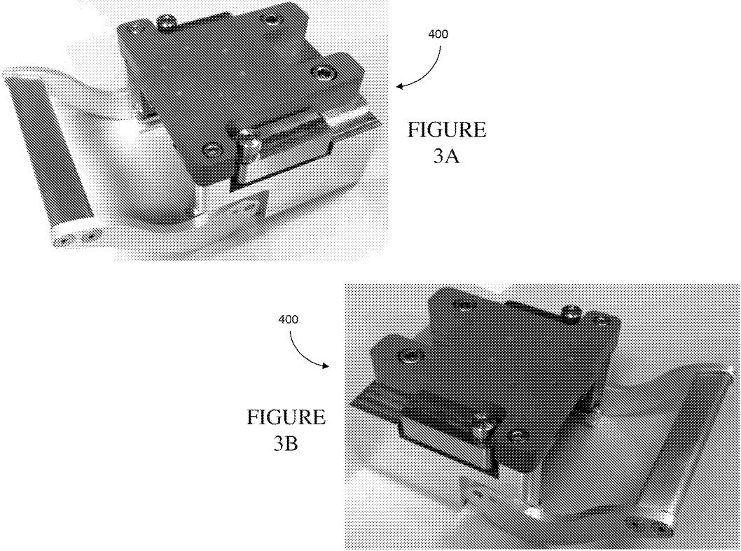

FIGS. 2A-2G illustrate embodiments of the replaceable cartridge 200 and cartridge 300. As discussed above, the cartridge 200, 300 contains the disinfectant/sterilant that is used by the system for reducing microorganisms on a surface 100 to disinfect, sterilize, and/or sanitize an item placed in the chamber 102. In some embodiments, the cartridge 200, 300 can contain a hydrogen peroxide solution. In particular, the cartridge 200, 300 contains a 50% hydrogen peroxide solution. In some embodiments, the cartridge 200, 300 can hold a volume between system 600 mL and 660 mL. In some examples, the cartridge 200, 300 can hold a volume of disinfectant/sterilant that is 600 mL, 605 mL, 610 mL, 615 mL, 620 mL, 625 mL, 630 mL, 635 mL, 640 mL, 645 mL, 650 mL, 660 mL between 600 mL and 605 mL, between 605 mL and 610 mL, between 610 and 615 mL, between 615 mL and 620 mL, between 620 mL and 625 mL, between 625 mL and 630 mL, between 630 mL and 635 mL, between 635 mL and 640 mL, between 640 mL and 645 mL, between 645 mL and 650 mL, between 650 mL and 655 mL, and between 655 mL and 660 mL.

As shown in FIGS. 2A-2B, the cartridge 200 can include a lid 210 that seals the cartridge 200 and prevents disinfectant/sterilant from spilling out of the cartridge 200. The lid 210 is configured to engage with the closure 220 and the spout 230 of the cartridge 200.

FIGS. 2C-2G illustrate another embodiment of the cartridge 300 in more detail. The cartridge 300 can include a body 302 and a closure 330 that engages with a proximal end of the body 302 to allow the disinfectant/sterilant in the body of the cartridge 300 to be dispensed into the system 100. In some embodiments, the cartridge 300 has a height 300h of between 4.0 - 5.0 inches. In some embodiments, the height 300 h is 4.0 inches, 4.1 inches, 4.2 inches, 4.3 inches, 4.4 inches, 4.5 inches, 4.6 inches, 4.7 inches, 4.8 inches, 4.9 inches, 5.0 inches or between about 4.0 - 4.1 inches, between about 4.1 - 4.2 inches, between about 4.2 - 4.3 inches, between about 4.3 - 4.4 inches, between about 4.4 - 4.5 inches, between about 4.5 - 4.6 inches, between about 4.6 - 4.7 inches, between about 4.7 - 4.8 inches, between about 4.8 - 4.9 inches, or between 4.9 - 5.0 inches. In some embodiments, the body 302 has a height 302h of between 3.0 - 4.0 inches. In some embodiments, the height 302h is 3.0 inches, 3.05 inches, 3.10 inches, 3.15 inches, 3.20 inches, 3.25 inches, 3.30 inches, 3.35 inches, 3.40 inches, 3.45 inches, 3.50 inches, 3.55 inches, 3.60 inches, 3.65 inches, 3.70 inches, 3.75 inches, 3.80 inches, 3.85 inches, 3.90 inches, 3.95 inches, 4.0 inches or between 3.0 - 3.10 inches, between 3.10 - 3.20 inches, between 3.20 - 3.30 inches, between 3.30 - 3.40 inches, between 3.40 - 3.50 inches, between 3.50 - 3.60 inches, between 3.60 - 3.70 inches, between 3.70 - 3.80 inches, between 3.80 - 3.90 inches, and between 3.90 - 4.0 inches.

In some examples, the body 302 of the cartridge 300 can include a bottle 310 and a bottle stand 320. As illustrated in the cross-sectional view of the cartridge 300 in FIG. 2G, the bottle stand 320 can include an opening 322 on the proximal end of the bottle stand 320 that is configured to receive and stabilize a distal end of the bottle 310. In some embodiments, the opening 322 has a cross-section that forms a taper and the distal end of the bottle 310 forms a corresponding taper. In some examples, the bottle 310 and bottle stand 320 are attached. In some embodiments, the bottle 310 and the bottle stand 320 are separate components that can engage and be secured with each other.

The bottle 310 can be configured to store and provide a volume of disinfectant/sterilant for the system for reducing microorganisms on a surface 100. In some embodiments, the outer surface of the bottle 310 can include a ribbed feature 380. The ribbed feature 380 can allow the bottle 310 to be more easily gripped by the user. In some embodiments, the ribbed feature 380 can be located on at least one outside surface of the bottle 310. In some examples, the ribbed feature 380 can be located on opposite sides on the outside surface of the bottle 310. The bottle 310 can include a neck 350 on a proximal end of the bottle 310. The neck 350 can form an opening to the bottle 310 and can have a smaller diameter than the diameter of the bottle 310. In some examples, the neck 350 can include a shoulder 354 that is disposed about the outer surface of the neck 350. The neck 350 can be configured to allow the cartridge 300 to be retained within the chamber 130 of the system 100. In some embodiments, the shoulder 354 can have a shoulder thickness 354h of between 0 - 0.5 inches. In some examples, the shoulder thickness 354h is 0 inches, 0.05 inches, 0.10 inches, 0.15 inches, 0.20 inches, 0.25 inches, 0.30 inches, 0.35 inches, 0.40 inches, 0.45 inches, 0.50 inches or between 0 - 0.05 inches, between 0.05 - 0.10 inches, between 0.10 -0.15 inches, between 0.15 -0.20 inches, between 0.20 -0.25 inches, between 0.25 - 0.30 inches, between 0.30 - 0.35 inches, between 0.35 - 0.40 inches, between 0.40 - 0.45 inches, or between 0.45 - 0.50 inches.

The cartridge 300 can include a closure 330 that is configured to engage with the neck 350 of the bottle 310. The closure 330 can include an inner threading 334 that is configured to threadingly engage with an external threading 352 on the neck 350 of the bottle 310. The exterior surface of the closure 330 can include a plurality of ridges that allow the closure 330 to be more easily gripped by the user. The closure 330 can include a receiving portion 332 on a proximal end of the closure 330. The receiving portion 332 can be centered on a top surface of the closure 330 and have a diameter that is smaller than the closure 330. In some embodiments, the receiving portion 332 can be configured to receive a spout 340. The spout 340 can include an opening 342 that extends through the center of the spout 340. The spout 340 can have a first end 344 and a second end 346. The first end 344 can have a greater diameter than the second end 346. The larger diameter of the first end 344 forms a lip that extends beyond the circumference of the receiving portion 332. As will be discussed in more detail below, the first end 344 of the spout 340 can be engaged such that the spout 340 moves in a proximal direction within the receiving portion 332 of the closure 330 into a first position. In this first position, the opening 342 of the spout 340 can be unsealed to allow the flow of disinfectant/sterilant out of the cartridge 300. In some examples, the first end 344 of the spout 340 can be engaged such that the spout 340 moves in a distal direction within the receiving portion 332 of the closure 330 into a second position. In some embodiments, when in this second position, the opening 342 of the spout 340 can be sealed to prevent the flow of disinfectant/sterilant out of the cartridge 300.

FIGS. 2F-2G illustrate a cross-sectional view of the cartridge 300. FIG. 2G illustrates cross-section A-A which is a lateral cross-section through a center of the cartridge 300. FIG. 2F illustrates an enlarged view of the proximal end of cross-section A-A. FIG. 2F illustrates the spout 340 in the second closed position, while FIG. 2G illustrates the spout 340 in the first opened position. The cartridge 300 can include an assembly 390 that secures the components of the cartridge 300 that allow disinfectant/sterilant to be dispensed from the cartridge 300. In some examples, the cartridge 300 includes a filter disk 360 that is circular and secured between an inner top surface of the closure 330 and a top of the opening of the neck 350 of the bottle 310.

In some embodiments, the assembly 390 includes a sump tube adapter 372 and a sump tubing 370. The sump tube adapter 372 can be configured to retain and position the spout 340 and the sump tubing 370 within the cartridge 300. As shown in FIGS. 2F-2G, the spout 340 can include a first end 372a and a second end 372b. The first end 372a can have a greater diameter than the second end 372b. The first end 372a can be configured to receive a securement portion 348 of the spout 340. The securement portion 348 can be located distal to the second end 346 of the spout 340. In some embodiments, the securement portion 348 limits the proximal movement of the spout 340 out of the receiving portion 332 and retains the spout 340 within the closure 330. In some examples, the second end 372b of the sump tube adapter 372 is configured to retain the sump tubing 370. As shown in FIG. 2G, in some embodiments, the sump tubing 370 can extend to the distal-most tip of the bottle 310. The sump tubing 370 can be fluidly connected to the opening 342 of the spout 340. This allow disinfectant/sterilant to be drawn out of the cartridge 300, through the sump tubing 370, and out of the cartridge 300 through the opening 342 of the spout 340. In some examples, the extension of the sump tubing 370 to the base of the tapered portion of the bottle 310 maximizes the volume of disinfectant/sterilant in the cartridge 300 dispensed. In some embodiments, the proximal end of the sump tubing 370.

FIGS. 2H-2I illustrate an enlarged cross-sectional view of the cartridge spout. FIG. 2H illustrates an embodiment of the spout 340 of the cartridge 300 when the spout 340 is in a first opened configuration. FIG. 2I illustrates an embodiment of the spout 340 of the cartridge 300 when the spout 340 is in a second closed configuration. As shown in FIGS. 2H-2I, the spout 340 is positioned within the receiving portion 332 of the closure 330. The spout 340 can be lifted out of the receiving portion 332 to allow fluid flow out of the cartridge 300. As discussed above, the spout 340 can include a first end 344 and a second end 346. The first end 344 forms a lip that has a greater diameter than the receiving portion 332 of the closure 330 to limit how far down the spout 340 can move within the receiving portion 332. The second end 346 can include a first portion that has a diameter that can move within the receiving portion 332. The spout 340 can also include a securement portion 348 that extends distally from the second end 346. The securement portion 348 can be configured to engage a lip 331 that extends from an inner surface of the receiving portion 332 to limit the movement of the spout 340 within the receiving portion 332 of the closure 330. In some embodiments, the securement portion 348 includes a primary snap hook 348a and a secondary snap hook 348b. As shown in FIG. 2I, the primary snap hook 348a can engage with the lip 331 to secure the spout 340 within the receiving portion 332 to keep the spout 340 in the second closed configuration to keep the cartridge 300 closed. In some embodiments, the primary snap hooks 348a of the spout 340 can provide enough retention force to discourage opening the cartridge 300 by any other method than with an engagement mechanism 400 (disclosed in more detail below). As shown in FIG. 2H, the secondary snap hook 348b can engage with the lip 331 to secure the spout 340 within the receiving portion 332 to position the spout 340 in a first opened position and prevent the spout 340 from extending open too far. In some embodiments, the closure 330 can include a filter disk 360 that is positioned on an underside of the closure 330. The filter disk 360 can serve as a cap vent liner and can be made of an ePTFE material (or other suitable material, including other polymeric materials). The filter disk 360 can allow gasses to escape but seal in the liquid. This can be important because hydrogen peroxide decomposes into water and oxygen gas. The oxygen gas must be vented to prevent buildup of pressure within the cartridge 300.

As discussed above, the spout 340 can include an opening 342 forming a channel that extends through the spout 340. As shown in FIG. 2H, the channel 342 can include a proximal end 342a and a distal end 342b. In some examples, the proximal end 342a has a smaller diameter than the distal end 342b. As shown in FIG. 2I, when the spout 340 is in the second closed position, the opening 342 of the spout 340 can be sealed on the conical seat 336. The conical seat 336 of the closure 330 can include a proximal end 336a and a distal end 336b. When the spout 340 is in the second closed position, the proximal end 336a is secured within the proximal end 342a of the opening 342 and the distal end 336b is secured within the distal end 342b to prevent fluid flow out of the closure 330 of the cartridge 300. As will be discussed in more detail below, the proximal end 336a of the conical seat 336 has a diameter that is less than the internal diameter of the distal end 336b of the conical seat 336. This can allow fluid to flow through the distal end 336b and around the proximal end 336a out of the opening 342 when the spout 340 is in a first opened position.

In some embodiments, the internal surface of the receiving portion 332 can include a plurality of sealing ribs 333 to engage with an outer surface of the second end 346 of the spout 340. The outer surface of the distal end 336b can include a plurality of sealing ribs 335 to engage an inner surface of the distal end 342b. The sealing ribs 333 and the sealing ribs 335 can allow the closure 330 and the spout 340 to provide sealing surfaces.

The closure 330 of the cartridge 300 can be configured to allow a user to remove the closure 330 from the bottle 310. As illustrated in FIG. 2J, the closure 330 can include a pull ring 338 and a plurality of thin areas 338a. The thin areas 338a can tear when the pull ring 338 is pulled or twisted with sufficient force. As shown in FIG. 2K, the closure 330 can include a plurality of engagement hooks 339a that secure the closure 330 to the bottle 310. When the pull ring 338 is pulled, the pull ring 338 can also release one of two of the engagement hooks 339a. Once the pull ring 338 is pulled, the closure 330 can be removed from the cartridge 300. To properly dispose of the cartridge 300, the cartridge 300 may need to be rinsed out to dilute any remaining peroxide residue in the bottle 310 prior to disposal.

In some embodiments, the pulling of the pull ring 338 and removing of the closure 330 can render the cartridge 300 unable to be reused in the disclosed system. In some embodiments, the engagement hooks 339a engage at a steep angle to the bottle 310 to prevent the closure 330 to be pried off the bottle 310 and increase the tamper-resistance of the cartridge 300. This can prevent improper refilling of the bottle 310. In some embodiments, the closure 330 can include a weak edge 339b that is positioned around the base of the closure 330. In some embodiments, the base of the closure 330 can be close enough to the bottle 310 to prevent the insertion of tools used to pry the closure 330 off the bottle 310 of the cartridge 300. In some examples, the weak edge 339b can deflect and deform easily if a prying tool is inserted, which can reduce the force a prying tool may apply on lifting the closure 330 from the bottle 310.

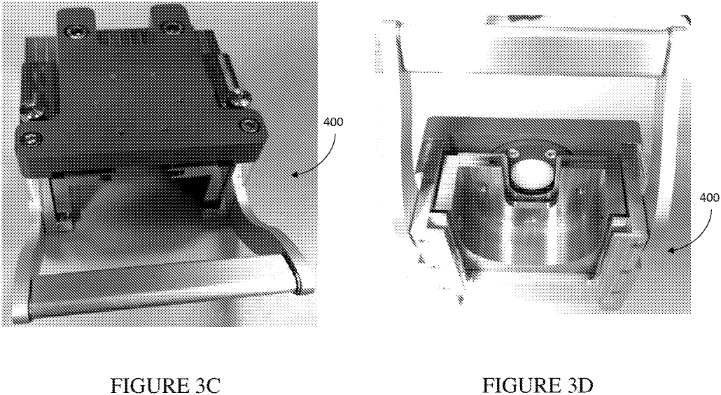

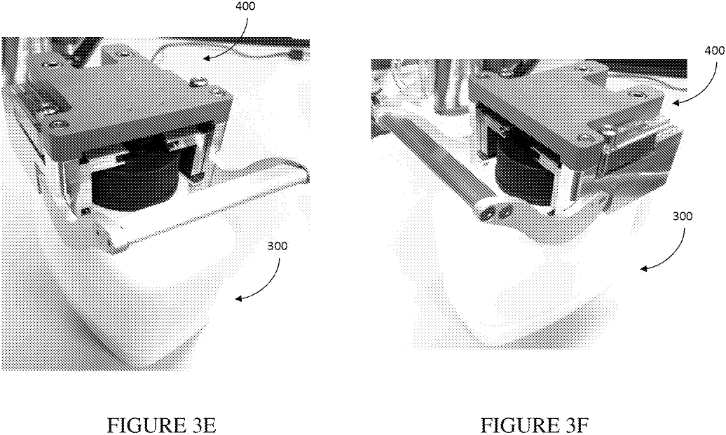

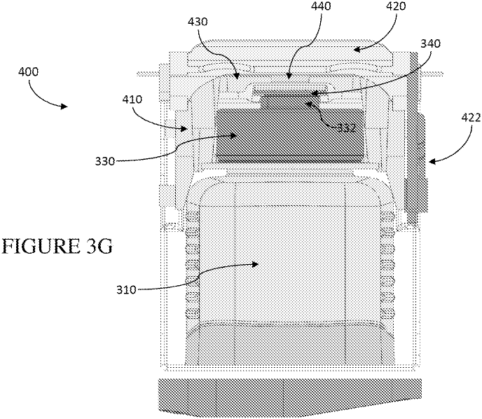

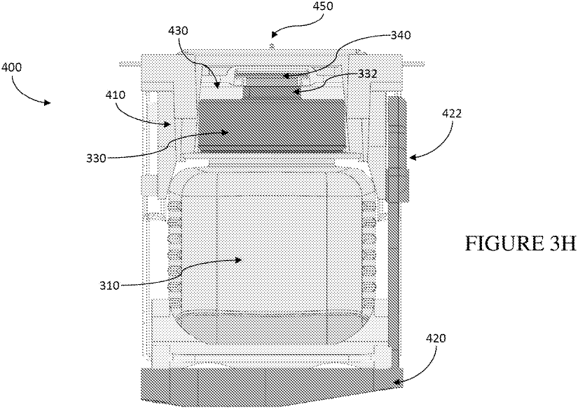

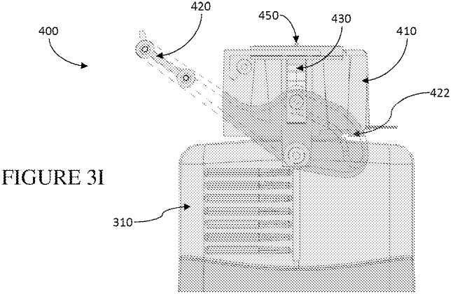

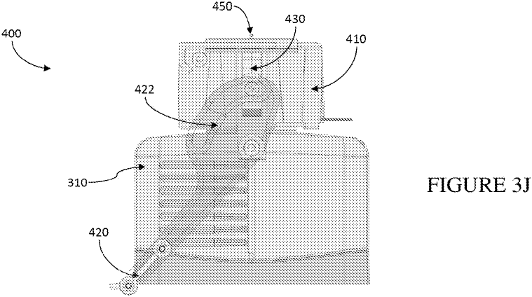

FIGS. 3A-3F illustrates an embodiment of an engagement mechanism 400 for securing the cartridge 300 in the system 100. FIGS. 3A-3D illustrates the engagement mechanism 400 in a first position while FIGS. 3E-3F illustrate two views of the engagement mechanism 400 engaged with a proximal end of the cartridge 300. In some embodiments, the engagement mechanism 400 is configured to move the spout 340 in the receiving portion 332 of the closure 330 to move the spout 340 from a closed position to an open position.

FIGS. 3G-3J illustrates an embodiment of the engagement mechanism 400. The engagement mechanism 400 can be configured to open and close the bottle 310 of the cartridge 300. When the bottle 310 is opened, the engagement mechanism 400 can allow suction of fluid from the bottle 310 into the rest of the system for disinfection/sterilization. FIGS. 3G and 3I illustrate the engagement mechanism 400 when the spout 340 in the closure 330 is in a closed position. FIGS. 3H and 3K illustrate the engagement mechanism 400 when the spout 340 in the closure 330 is in an opened position. The engagement mechanism 400 can include a frame 410 with a spout lifter 430 that is configured to engage the spout 340 of the cartridge 300.

The engagement mechanism 400 can include a handle 420 and a plurality of camming arms 422 that can move spout lifter 430 in the engagement mechanism 400 between a closed and an opened position. The engagement mechanism 400 can include a flow seal 440 with a flow fitting 450 that can allow be fluidly connected to the spout 340 such that fluid can flow out of the cartridge 300. As shown in FIGS. 3G and 3I, the spout lifter 430 can be secured to the first end 344 of the spout 340. The spout lifter 430 can engage with the portion of the first end 344 that extends past the receiving portion 332. The handle 420 and the plurality of camming arms 422 can be in a first raised position while the spout 340 is in the closed position. As shown in FIG. 3G, a gap exists between the spout 340 and the flow seal 440.

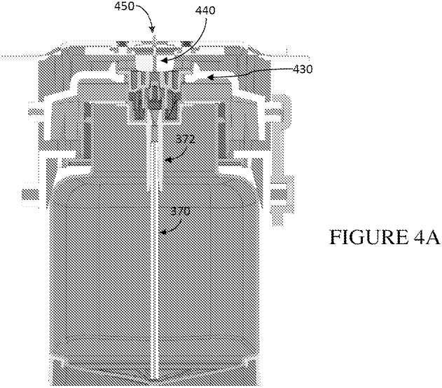

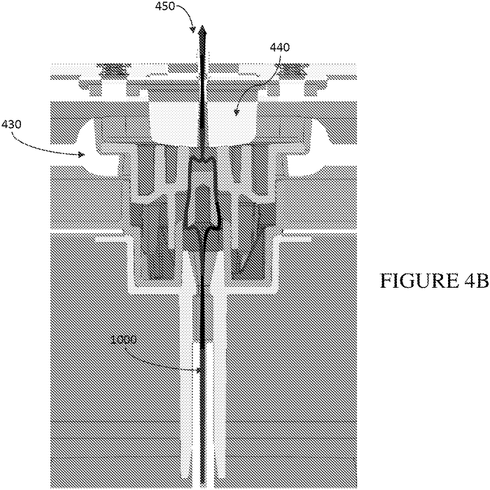

To move the spout 340 into an opened position, the handle 420 can be actuated and moved into a second lowered position as illustrated in FIG. 3J. A camming surface on the camming arm 422 drives the spout lifter 430 upward. The spout lifter 430 unseats the spout 340 from the closure 330 and seals it against the flow seal 440. As shown in FIG. 3H, as the handle 420 is actuated, the spout lifter 430 moves upwardly to lift the spout 340 out of the receiving portion 332. As shown in FIG. 2I, lifting the spout 340 out of the receiving portion 332, shifts the proximal end 336a of the conical seat 336 into the distal end 342b of the channel of the opening 342. The proximal end 336a can be cone shaped to allow fluid to flow around the proximal end 336a and out of the opening 342. As will be discussed in more detail below, FIGS. 4A-4B show the position of the spout 340 relative to the flow seal 440 and the flow fitting 450. When the spout 340 is moved out of the receiving portion 332 and into an open position, the spout 340 is moved adjacent to a bottom surface of the flow seal 440 to seal the spout 340 against the flow seal 440. This can allow the spout 340 to be fluidly connected to the flow seal 440 to allow fluid to flow out of the cartridge 300 and out of the engagement mechanism 400 through the flow fitting 450.

In some embodiments, when the handle 420 is lifted, an opposite camming surface presses the spout 340 down into the closure 330, moving the closure 330 into a closed position and sealing the bottle 310. In some embodiments, near the top of the handle 420 motion, it shifts the spout lifter 430 up against a small amount to releasing pressure on the spout 340. In some examples, this motion can help to release the cartridge 300 from the engagement mechanism 400.

FIGS. 4A-4B illustrate an embodiment of the fluid flow out of the cartridge 300 and engagement mechanism 400 when the engagement mechanism 400 engages with the spout 340 such that the spout 340 is in an open configuration. As shown in FIG. 4A, the tubing 370, the tube adapter 372, the closure 330, the spout 340, the flow seal 440 and the flow fitting 450 are fluidly connected. FIG. 4B illustrates the fluid flow 1000 when suction is applied to the engagement mechanism 400 engaged with the cartridge 300. The suction on the flow fitting 450 can draw liquid up from the bottle 310 through the tubing 370, the tube adapter 372, the closure 330, the spout 340, the flow seal 440, and up to the flow fitting 450.

### Evaporator/Nebulizer

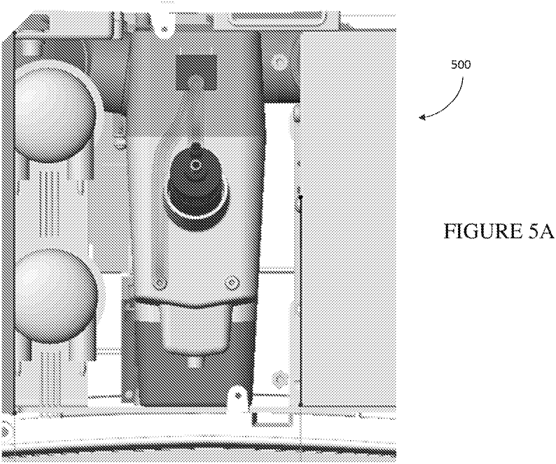

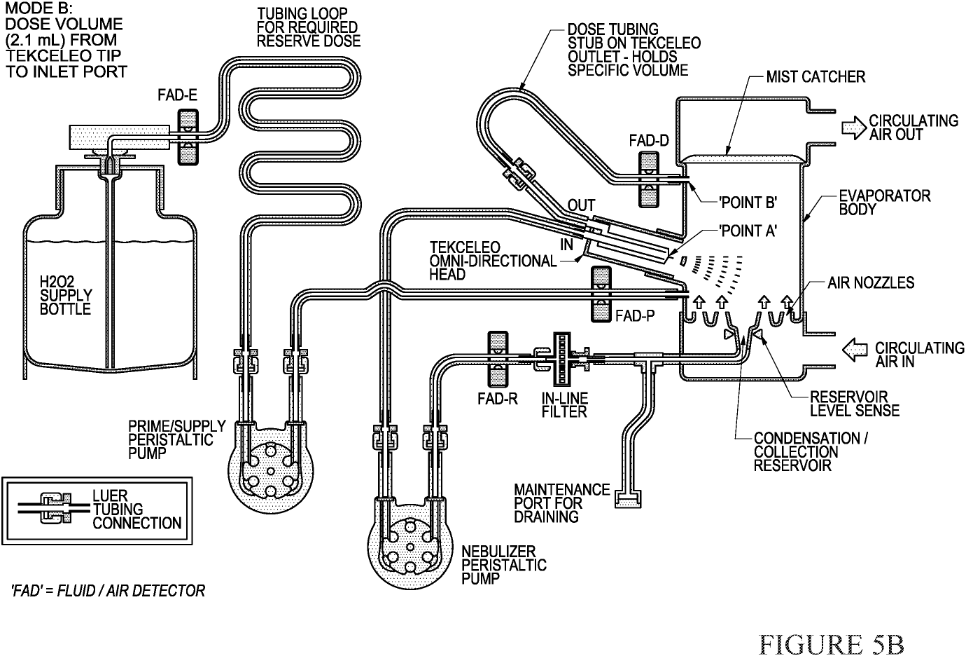

In some embodiments, the system for reducing the viability of microorganisms on a surface 100 can include a nebulizer or an evaporator. FIGS. 5A-5B illustrate an embodiment of a nebulizer or an evaporator for use in the system 100. As shown in FIG. 5B, the nebulizer or an evaporator can be configured to engage with the cartridge 300 to provide disinfectant/sterilant (i.e., the H₂O₂ solution) into the chamber 102 of the system 100. In some embodiments, the nebulizer or an evaporator can be fluidly connected to the cartridge 300 such that the cartridge 300 can be dispensed from the nebulizer 630 as a spray. In some examples the spray of disinfectant/sterilant is deposited on the surface of the at least one item 101 in the chamber 102.

In some examples, the nebulizer can be programmed to deliver 0.01 mL of 50% H₂O₂ per second for 3.5 minutes, yielding 2.1 mL of liquid H₂O₂ to the nebulizer 630 for each disinfection cycle. Greater or lesser amounts or rates can be used, depending on the embodiment.

### Ozone Generator

The system for reducing the viability of microorganisms on a surface 100 can include an ozone generator. In several embodiments, a dielectric barrier discharge system is used. The plasma free radical generator 30 can be any kind of dielectric barrier discharge device, electrical corona device, a glow discharge device, or a microwave generator. One non-limiting example of a device which can be used within the teachings of the disclosure is an ozone generator such as, for example, ozone generator cell SY-G20 manufactured by Longma Industrial Zone, Bao’an District, Shenzhen, 518108, P.R.C. In some embodiments, the ozone generator includes two plates, each of which is configured to provide approximately 5 gm/hour600 ppm/min per plate. In some embodiments, the ozone generator is a Dielectric Barrier Discharge ozone generator wherein the metal is not exposed. Depending on the embodiment, any other type of system that generates free radicals may be used, for example a system or device that produces sufficient energy to break bonds, such as covalent bonds, for example through hemolytic bond cleavage. Additional embodiments, employ free radical generators that operate via silent corona discharge UV light to split O2 to create single oxygen atoms, which then interact with O2 to form O3 (ozone).

In some embodiments, the ozone generator is a low pressure mercury ozone generator. In some examples, a low pressure mercury ozone generator can be configured to be used in water ozonation application and can have good bactericidal properties.

In some embodiments, the ozone generator is a Xe2 Excimer ozone generator. In some examples, the Xe2 Excimer ozone generator can be configured to have significantly better ozone generation efficiency (e.g. 40%) and the amount of nitric acid produced can be 0.2 ppm which is approximately 100 times lower than using DBD ozone generator. In some embodiments, the small amount of nitric acid produced can improve material compatibility significantly.

It will be understood by persons skilled in the art that, unless otherwise specifically indicted, references to an “ozone generator” does not refer any specific structure or device for ozone production. Ozone generator can refer to any structure, device, or combination of structures or devices that results in the direct or indirect production of ozone for the purposes of the disclosed systems. For example, the “ozone generator” can be a device that only produces ozone or it can refer to a device such as a plasma generator that produces ozone as a byproduct.

### Filters

The system for reducing microorganisms on a surface 100 can include at least one filter in the inlet and/or the outlet. A variety of filter types can be used, depending on the embodiment. For example, in several embodiments, a HEPA filter is used. In some embodiments, ionic filters, carbon filters, UV filters, cellulose filters, silica based filters or the like are used, either alone or in combination. In some embodiments, the filter can be configured to filter environmental air and allow it to pass into the system.

### Pumps

In some embodiments, the system for reducing microorganisms on a surface 100 can include at least one pump. In some examples, the pump can be a peristaltic pump. In some embodiments, the peristaltic pump can be configured to deliver the disinfectant solution (e.g., 50% H₂O₂ solution) to the nebulizer. In several embodiments, the use of a peristaltic pump allows a precise amount of the disinfectant solution to be delivered to the nebulizer in a controlled manner, thus ensuring a consistent amount of disinfectant solution is introduced into the chamber at a given point in the cycle.

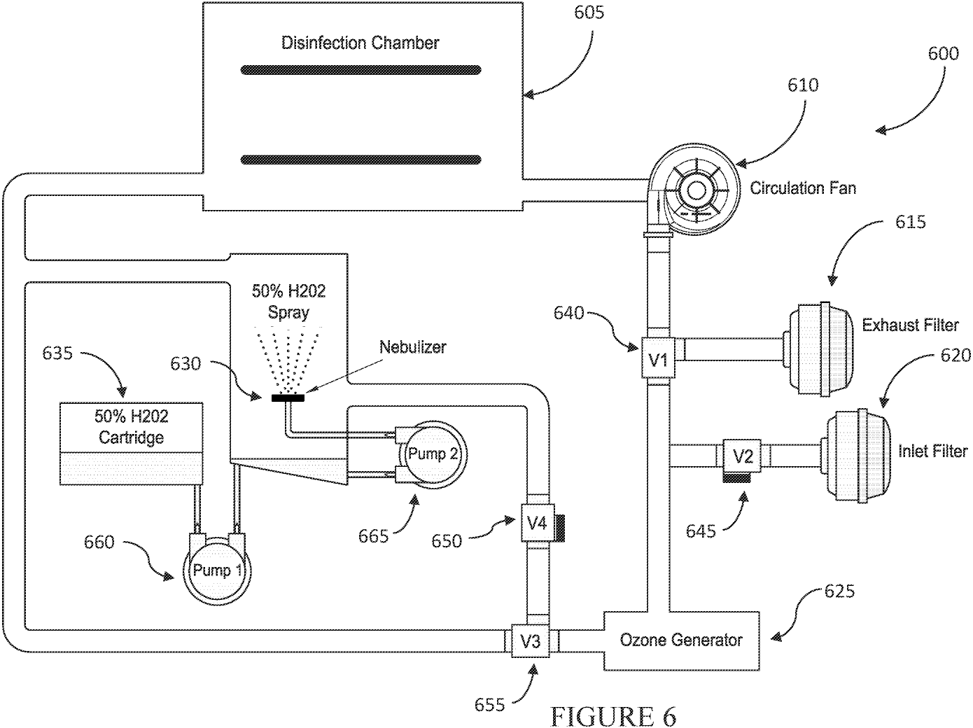

As will be described in more detail below, in some embodiments, a system for disinfection/sterilization can include a plurality of pumps. The embodiments illustrated in FIGS. 6-11 include two pumps that deliver disinfectant/sterilant from a cartridge to a nebulizer. As will be discussed, one pump draws the liquid from the cartridge and fills a reservoir of a nebulizer while a second pump pumps disinfectant/sterilant to the nebulizer. In additional embodiments, a single pump performs both functions.

### Fans

The system for reducing the viability of microorganisms on a surface 100 can include at least one circulating fan. In some embodiments, the fan can instead be a blower or air distribution unit which is used to convey gases through the system 100. In several embodiments, this unit also provides sufficient motive force to push the disinfectant/sterilant into the chamber. In some embodiments, the blower/flow generator comprises a pump, such as a circulating pump, a positive displacement pump, or an air conveyor, a fan, or a blower optionally integrated with a flow distributor. In embodiments comprising a flow distributor, the distributor is configured to convey a desired percentage of air/disinfectant/sterilant to either the ozone generator and/or the nebulizer. As discussed in more detail below, the ratio of air/disinfectant/sterilant conveyed to the ozone generator and nebulizer is variable, depending on the embodiment. In some embodiments, the variation in flow is fixed prior to a disinfection, sterilization, and/or sanitization cycle. In additional embodiments, the variation in flow can be dynamic during a cycle, for example, adjusting the flow between the ozone generator and the nebulizer depending on the amount of disinfectant/sterilant is being recycled from the chamber.

### Monitoring Systems

The system for reducing the viability of microorganisms on a surface 100 can include integrated software and monitoring sensors to control each of the components in the system 100 and the amount of disinfectant/sterilant dispensed.

In some embodiments, the system 100 can be configured to monitor the operation of the ozone generator. For example, a monitoring system of the system 100 is configured to monitor the electrical current provided to the ozone generator 625 to ensure that the electrical current is within a prescribed range to ensure proper operating conditions.

In some examples, the system 100 can be configured to monitor the operation of the nebulizer. For example, a monitoring system of the system 100 can be configured to monitor the electrical voltage and the electrical current provided to the nebulizer 630 to ensure that the electrical voltage and the electrical current are within a prescribed range to ensure proper operating conditions.

In some embodiments, the system 100 can be configured to monitor the operation of the at least one pumps. For example, a monitoring system of the system 100 can be configured to monitor the electrical voltage and the electrical current provided to the pump(s) (e.g., peristaltic pump(s)) to ensure that the electrical voltage and the electrical current are within a prescribed range to ensure proper operating conditions. In some examples, the monitoring system of the system 100 is programmed to ensure that the at least one pump delivers a specified amount of H₂O₂ to the nebulizer. In some embodiments, the at least one peristaltic pump can be programmed to ensure the specified volume of H₂O₂ is delivered to the nebulizer. In some examples, the system 100 can be configured to measure the H₂O₂ usage through a gravimetric method in order to indicate the volume of H₂O₂ delivered by the at least one pump to the nebulizer during the disinfection process.

In some examples, the system 100 can be configured to monitor the operation of the circulating fan. For example, a monitoring system of the system 100 can be configured to monitor the electrical voltage and the electrical current provided to the circulating fan to ensure that the electrical voltage and the electrical current are within a prescribed range to ensure proper operating conditions. In some embodiments, the system 100 can be configured ensure that the circulating fan is operating at a fixed speed.

In some embodiments, the system 100 can be configured to monitor the operation of the cartridge for disinfectant/sterilant. For example, a monitoring system of the system 100 can be configured to monitor the volume within the cartridge 300 by an electronic sensor.

In some examples, the system 100 can be configured to monitor the operation of the at least one filter. For example, a monitoring system of the system 100 can include a carbon filter and integrated emissions sensor to monitor the at least one filter.

In some embodiments, the system 100 can include software that is programmed to operate the monitoring system described above. In some examples, if any system fault are detected above, the software for the system 100 can generate an appropriate error code that is displayed to the user on the screen 150. In some embodiments, the system software can display on the screen 150 instructions and/or next steps to revolve the identified error.

### Method for Disinfection, Sanitization, and/or Sterilization

As discussed above, in some embodiments, the system for reducing the viability of microorganisms on a surface 100 takes a standardized approach to disinfection while using a well-recognized hospital disinfectant, hydrogen peroxide, which rapidly penetrates cell walls and kills microbes. The system 100 can be configured to circulate disinfectant/sterilant throughout the chamber 102 to uniformly treat difficult to reach surfaces. Once an item is placed inside the chamber 102, the single button operation of the system 100 can make infection prevention simple. As will be discussed in more detail below, the disclosed system 100 is configured to provide disinfection, sterilization, and/or sanitization with minimal user input and without damaging the item(s) placed in the chamber 102 for reducing microorganisms on a surface. In some embodiments, the system 100 provides a method for disinfection, sanitization, and/or sterilization 700 that does not use high heat or pressure and does not damage the item(s) being disinfected, sterilized, and/or sanitized. In some embodiments, the system 100 provides a method 700 that minimizes the waste produced. In some embodiments, the system 100 provides a method 700 that disinfects, sterilizes, and/or sanitizes without harmful residue, emissions, byproducts, or exposing the user to the disinfectant/sterilant used. The method 700 that will be discussed in more detail below is configured to replace manual and variable disinfection, sterilization, and/or sanitization processes with an automated and standardized exposure-free system.

In the systems discussed below, the disclosed system 600 and method 700 for reducing microorganisms on a surface is fully automated and can disinfect hard non-porous surfaces of reusable medical devices and general use items used in healthcare facilities.

In some embodiments, the disclosed systems operate at ambient temperature and ambient pressure conditions in a continuous closed loop flow throughout the cycle. The user may be required to clean and dry items prior to placing the item for disinfection into the system.

In some embodiments, more than one item can be disinfected at the same time. To disinfect more than one item, the items can be loaded into the chamber and placed such that items to be disinfected are not in contact with each other and no portion of any item overlaps with itself. The item can be required not to touch any of the disinfection chamber walls.

As will be discussed in further detail below, in some embodiments, the disclosed disinfection/sterilization systems does not include a heater. The disinfection/sterilization of the surface of the at least one medical devices and/or items is conducted at an ambient or approximately ambient temperature (e.g., some temperature increase may occur due to heat generated by, for example blower motors).

In some examples, the disclosed systems for disinfection/sterilization do not include a dehumidifier. The systems include a purging phase wherein the internal fluid flow is flushed and replaced with fresh air from the surrounding environment.

In some examples, the blowers can keep the system under pressure (i.e., below ambient) such that the system does not create a vacuum internally. This can prevent disinfectant/sterilant from leaking out of the system if a leak develops. In some embodiments, the blower keeps the system at between about 3 kPascals to 15 kPascals, for example, no more than about 4.5 kPascals.

The below methods are discussed with regard to disinfection, however the disclosed systems (i.e., system 600 and system 900) can be used to achieve sterilization and/or sanitization. To achieve sterilization or sanitization, the below times for operation of the disclosed systems can be increased to ensure sufficient reduction of bacteria. For example, the operating times for the below method can be increased (e.g., doubled) to achieve sterilization instead of disinfection.

### Method for Disinfection, Sterilization, and/or Sanitization

FIG. 6 illustrates a schematic diagram of an embodiment of a system 600 for reducing microorganisms on a surface. As illustrated, the system 600 can be a fully integrated system which includes a chamber 605, an ozone generator 625, a nebulizer 630, at least one pump (i.e., pump 660 and pump 665), a circulating fan 610, a plurality of valves (i.e., valve 640, valve 645, valve 650, valve 655), an inlet and inlet filter 620, and an outlet and exhaust filter 615. In some embodiments, the at least one pump 655, 660 is a peristaltic pump.

As will be discussed in more detail below, the system 600 can be designed to draw a precise volume between about 1.5 mL and 2.5 mL (e.g., 2.1 mL) of liquid disinfectant (i.e., 50% H₂O₂) from the cartridge by the at least one pump 655, 660, nebulize the liquid into a spray using the nebulizer 630, and transport the spray via forced air in a continuous closed loop flow through the disinfection chamber 605. This disinfectant spray is configured to contact the surfaces of the items placed in the disinfection chamber 605 to inactivate the pathogens during the cycle. In some embodiments, the system 600 can be designed to draw a volume of 1.6 mL, 1.7 mL, 1.8 mL, 1.9 mL, 2.0 mL, 2.1 mL, 2.2 mL, 2.3 mL, 2.4 mL, 2.5 mL, between about 1.6 mL -1.7 mL, between about 1.7 mL -1.8 mL, between about 1.8 mL -1.9 mL, between about 1.9 mL -2.0 mL, between about 2.0 mL -2.1 mL, between about 2.1 mL -2.2 mL, between about 2.2 mL -2.3 mL, between about 2.3 mL -2.4 mL, between about 2.4 mL -2.5 mL and any value in between those ranges listed, including endpoints.

The system 600 can be configured to include an ozone generator 625. In some embodiments, the ozone generator 625 is configured to produce ozone. The ozone produced can be used in two ways in the system 600. In some embodiments, the ozone produced by the ozone generator 625 can be configured to precondition the disinfection chamber 605. In some embodiments, the ozone produced by the ozone generator 625 can be configured to neutralize the residual H₂O₂ after the item placed in the disinfection chamber 605 has been disinfected, sterilized, and/or sanitized.

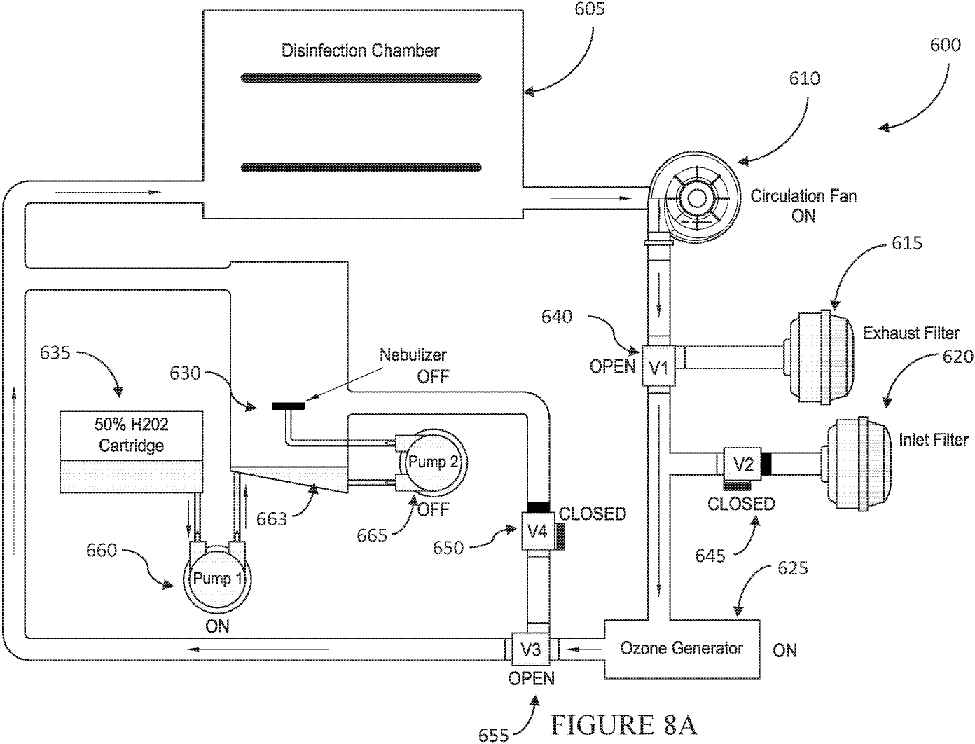

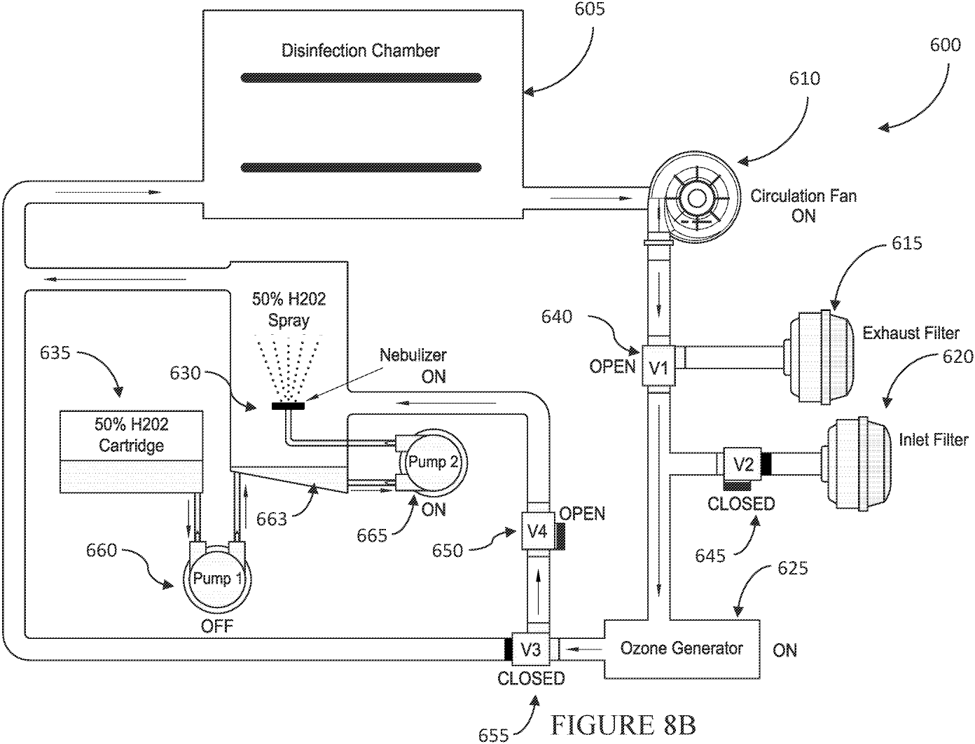

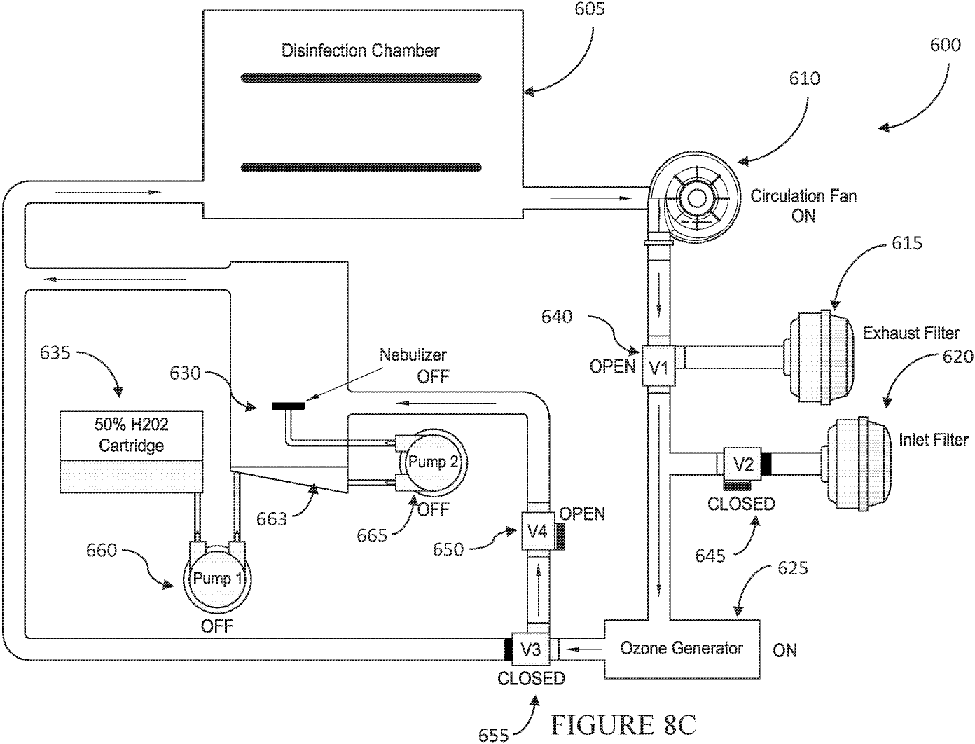

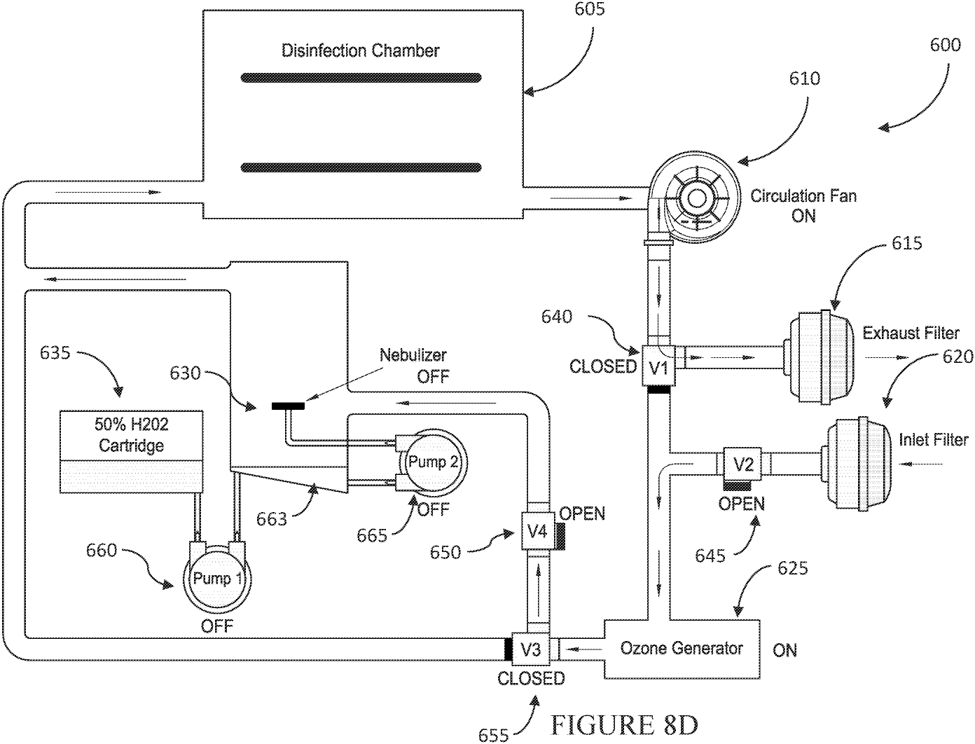

As shown in FIGS. 8A-8D, the system 600 includes a reservoir 663 between the pump 660 and the pump 665. The pump 660 can be preprogrammed to pump out a predetermined amount of disinfectant/sterilant until the cartridge 635 is empty. Because excess disinfectant/sterilant (e.g., H₂O₂) is removed after each disinfection/sterilization cycle, the pump 665 can be responsible for delivering the proper amount of disinfectant/sterilant (e.g., H₂O₂) to the nebulizer 630. Any excess amount of disinfectant/sterilant (e.g., H₂O₂) can be stored in the reservoir 663. In some embodiments, the reservoir 663 has a predetermined amount of disinfectant/sterilant (e.g., H₂O₂) that is stored; if the amount of disinfectant/sterilant (e.g., H₂O₂) falls below the predetermined amount, pump 660 will pump out disinfectant/sterilant to fill the reservoir 663 to the proper amount. In some examples, the volume stored in the reservoir 663 is the amount of disinfectant/sterilant that is delivered to the nebulizer 630. The pump 665 can be programmed to pump whatever is in the reservoir 663. However, if there is any disinfectant/sterilant unused during disinfection/sterilization, the disinfectant/sterilant will be stored in the reservoir 663. When the amount of disinfectant/sterilant of the reservoir 663 falls below the predetermined volume, the pump 660 will pump disinfectant/sterilant out of the cartridge 635 to fill the reservoir 663 to the predetermined volume.

As provided in the table below, the system 600 can be configured to disinfect an item. In some embodiments, the system 600 operates at ambient temperature and pressure conditions in a continuous closed loop flow throughout the cycle. In some embodiments, the system 600 is configured to operate at a temperature of 20° C., 21° C., 22° C., 23° C., 24° C., 25° C. In some embodiments, the system 600 is configured to operate at a temperature between about 20° C. and 21° C., between about 21° C. and 22° C., between about 22° C. and 23° C., between about 23° C. and 24° C., and between about 24° C. and 25° C. As discussed above, the disclosed system 600 can operate to disinfect without the use of a heater.

In some examples, the system 600 is configured to operate with a relative humidity of 20%, 25%, 30%, 35%, 40%, 45%, 50%, 55%, and 60%. In some embodiments, the system 600 is configured to operate with a relative humidity of between about 20% and 25%, between about 25% and 30%, between about 30% and 35%, between about 35% and 40%, between about 40% and 45%, between about 45% and 50%, between about 50% and 55%, between about 55% and 60%. As discussed above, the system 600 does not include a dehumidifier remove moisture from the system 600. The below table provides a summary of an embodiment of the range of operating conditions of the system 600:

| Operating Conditions | Minimum | Maximum |
|---|---|---|
| Ambient Temperature | 20° C. | 25° C. |
| Relative Humidity | 20% | 60% |

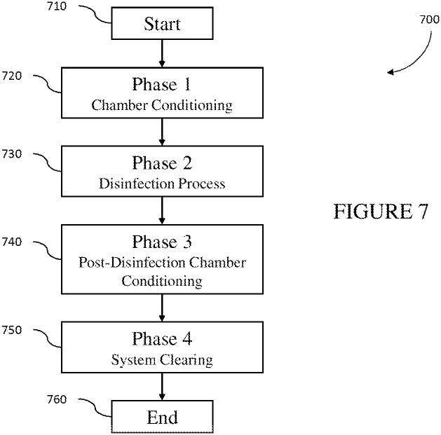

FIG. 7 illustrates a flowchart of a non-limiting method for disinfection, sanitization, and/or sterilization 700. In some examples, the method 700 is configured to operate in the system 600. The method 700 can start at step 710, Phase 1 at step 720, Phase 2 at step 730, Phase 3 at step 740, Phase 4 at step 750, and end at step 760. Each of these phases will be discussed in more detail below. In some embodiments, the process time for the method 700 is 10 minutes and can include four distinct phases. In some examples, the contact time with the disinfectant (i.e., H₂O₂) is approximately 4.5 minutes of the 10-minute process. A summary of the time for each phase of an embodiment of method 700 is provided below:

| Phase | Description | Phase Duration (min) | Elapsed Time (min) |
|---|---|---|---|
| Phase 1 | Chamber Conditioning | 2.5 | 0 - 2.5 |
| Phase 2 | Disinfection Process (contact time) | 4.5 | 2.5 - 7.0 |
| Phase 3 | Post-Disinfection Chamber Conditioning | 2.0 | 7.0 - 9.0 |
| Phase 4 | System Clearing | 1.0 | 9.0 - 10.0 |

In some embodiments, the Phase 1 Chamber Conditioning phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 -1.0 minutes, between about 1.0 -1.5 minutes, between about 1.5 -2.0 minutes, between about 2.0 -2.5 minutes, between about 2.5 -3.0 minutes, between about 3.0 -3.5 minutes, between about 3.5 -4.0 minutes, between about 4.0 -4.5 minutes, between about 4.5 -5.0 minutes and any value in between those ranges listed, including endpoints. In some embodiments, the Phase 2 Disinfection Process phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 - 1.0 minutes, between about 1.0 - 1.5 minutes, between about 1.5 -2.0 minutes, between about 2.0 -2.5 minutes, between about 2.5 -3.0 minutes, between about 3.0 - 3.5 minutes, between about 3.5 - 4.0 minutes, between about 4.0 - 4.5 minutes, between about 4.5 - 5.0 minutes and any value in between those ranges listed, including endpoints. In some embodiments, the Phase 3 Post-Disinfection Chamber Conditioning phase phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 - 1.0 minutes, between about 1.0 - 1.5 minutes, between about 1.5 - 2.0 minutes, between about 2.0 - 2.5 minutes, between about 2.5 - 3.0 minutes, between about 3.0 - 3.5 minutes, between about 3.5 - 4.0 minutes, between about 4.0 - 4.5 minutes, between about 4.5 - 5.0 minutes and any value in between those ranges listed, including endpoints. In some examples, the Phase 4 System Clearing can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 - 1.0 minutes, between about 1.0 -1.5 minutes, between about 1.5 - 2.0 minutes, between about 2.0 - 2.5 minutes, between about 2.5 - 3.0 minutes, between about 3.0 - 3.5 minutes, between about 3.5 - 4.0 minutes, between about 4.0 - 4.5 minutes, between about 4.5 - 5.0 minutes and any value in between those ranges listed, including endpoints.

In some embodiments, the method 700 can start at step 710. Prior to inserting the item(s) to be disinfected into the disinfection chamber 605, the user must first clean and dry the items to be placed in the disinfection chamber 605. Once the item is clean and dry, the user can place the item into the disinfection chamber 605.

The method 700 can include step 720 - Phase 1 Chamber Conditioning -wherein the disinfection chamber 605 is conditioned. In some embodiments, the chamber conditioning of step 720 can last for approximately 2.5 minutes. FIG. 8A provides an illustration of the system 600 during the chamber conditioning.

As the name suggests, during chamber conditioning of Phase 1, conditions the chamber for H₂O₂ disinfection by converting H₂O to OH radicals, thereby reducing residual moisture. In some embodiments, during the Chamber Conditioning of Phase 1, ozone is supplied by the ozone generator 625 to the disinfection chamber 605 through a closed loop flow. Phase 1 Chamber Conditioning can optimize the disinfection chamber 605 for disinfection. In some examples, the disinfection chamber 605 is optimized for H₂O₂ disinfection.

A non-limiting example of the status of the components of the system 600 during the Chamber Conditioning are provided below:

| | |
|---|---|
| Element | Status |
| circulating fan 610 | ON |
| valve 640 (V1) | OPEN (to internal circulation) |
| valve 645 (V2) | CLOSED (to inlet filter) |
| valve 655 (V3) | OPEN (to internal circulation) |
| valve 650 (V4) | CLOSED |
| pump 660 (Pump 1) | ON (fills the nebulizer reservoir) |
| pump 665 (Pump 2) | OFF |
| ozone generator 625 | ON - first 1.5 min of 2.5 min phase |
| | OFF - last 1.0 min of 2.5 min phase |

As illustrated in FIG. 8A, the circulation fan 610 is turned on to circulate air through the system 600. In some examples, the valve 640 to the exhaust filter 615 is opened, the valve 645 to the inlet filter 620 is closed, the valve 655 is opened, and the valve 650 is closed. In some embodiments, the pump 660 is “ON” which allows for the nebulizer reservoir to fill. As shown in FIG. 8A, as the pump 665 is turned “OFF”, the chamber conditioning in Phase 1 does not use any H₂O₂.

In some examples, the ozone generator 625 is turned on for part of the chamber conditioning phase of step 720. The ozone generator 625 can be turned on for the first part of the chamber conditioning phase of step 720. The ozone generator 625 can be turned on for the first 1.5 minutes of the 2.5 minute chamber conditioning phase. This can allow for ozone to be supplied to the disinfection chamber 605 from the ozone generator 625 for 1.5 minutes. As shown in FIG. 8A, each of the valves (i.e., valve 640, valve 645, valve 650, and valve 655) are positioned to allow continuous circulation of ozone through the disinfection chamber 605 in a closed loop flow. In particular, air flow occurs from the ozone generator 625 to the disinfection chamber 605 and from the disinfection chamber 605 to the ozone generator 625. The circulation of ozone during this phase conditions the chamber for H₂O₂ disinfection by converting H₂O molecules to OH radicals (disinfecting molecules) and thereby reducing residual moisture.

In some examples, the ozone generator 625 is turned off for a second part of the chamber conditioining phase. In some embodiments, the ozone generator 625 is turned off for the last 1.0 minute of the 2.5 minute chamber conditioning phase. In some embodiments, during the last 1.0 minute of the 2.5 minute chamber conditioning phase, the ozone generator 625 is turned off and the ozone level will decay over time as it interacts with surfaces within the system 600.

Method 700 can include step 730 - Phase 2 Disinfection Process -wherein an item placed in the disinfection chamber 605 is disinfected. In some examples, the Phase 2 Disinfection Process of step 730 can last for approximatly 4.5 minutes. FIG. 8B provides an illustration of the system 600 during the Phase 2 Disinfection Process.

During the Phase 2 Disinfection Process, the disinfectant is introduced into the disinfection chamber 605. In some embodiments, the disinfectant is a 50% hydrogen peroxide solution. The disinfectant can be introduced into the disinfection chamber 605 through a nebulizer 630. The nebulizer 630 can convert the disinfectant (i.e., the 50% hydrogen peroxide solution) from a liquid into a micro-spray that allows the disinfectant to move in the closed loop flow. In some embodiments, this micro-spray is the active ingredient used in the disinfection process.

A non-limiting example of the status of the components of the system 600 during the Disinfection Process is provided below:

| | |
|---|---|
| Element | Status |
| circulating fan 610 | ON |
| valve 640 (V1) | OPEN (to internal circulation, closed to exhaust filters) |
| valve 645 (V2) | CLOSED (to inlet filter) |
| valve 655 (V3) | CLOSED (to the chamber) |
| valve 650 (V4) | OPEN (to nebulizer chamber and internal circulation) |
| pump 660 (Pump 1) | OFF |
| pump 665 & nebulizer 630 (Pump 2 & Nebulizer) | ON (delivers 2.1 mL and nebulizes for 3.5 min.) |
| ozone generator 625 | OFF |

As illustrated in FIG. 8B, the circulating fan 610 is turned on to circulate air through the system 600. In some examples, the valve 640 to the internal circulation but closed to the exhaust filter 615, the valve 645 to the inlet filter 620 is closed, the valve 655 is closed to the disinfection chamber 605, and the valve 650 is opened to the internal circulation and the chamber of the nebulizer 630. In some embodiments, the pump 660 is “OFF” to prevent the disinfectant (e.g., 50% H₂O₂ solution) from filling the nebulizer 630. In some examples, the pump 665 and the nebulizer 630 are turned “ON” to deliver disinfectant through the nebulizer 630.

In some examples, 50% H₂O₂ is the active ingredient in the disinfection process of step 730. During the disinfection process, the pump 665 is a peristaltic pump that is fluidly connected to the nebulizer 630. In some embodiments, the nebulizer 630 is an 8-micron nebulizer mesh. The pump 665 can be configured to deliver approximately 2.1 mL of 50% H₂O₂ disinfectant to the nebulizer 630 for the first 3.5 minutes of the contact time. In some embodiments, the system 600 can be designed to deliver a volume of 1.6 mL, 1.7 mL, 1.8 mL, 1.9 mL, 2.0 mL, 2.1 mL, 2.2 mL, 2.3 mL, 2.4 mL, 2.5 mL, between about 1.6 mL - 1.7 mL, between about 1.7 mL - 1.8 mL, between about 1.8 mL -1.9 mL, between about 1.9 mL - 2.0 mL, between about 2.0 mL - 2.1 mL, between about 2.1 mL -2.2 mL, between about 2.2 mL -2.3 mL, between about 2.3 mL - 2.4 mL, between about 2.4 mL - 2.5 mL and any value in between those ranges listed, including endpoints. In some embodiments, the peristaltic pump 665 is programmed to deliver 0.01 mL of 50% H₂O₂ per second for 3.5 minutes (210 seconds) to the nebulizer 630. The H₂O₂ solution can then be nebulized into a spray and be continuously circulated through the disinfection chamber 605 in a closed flow loop. As shown in FIG. 8B, each of the valves (i.e., valve 640, valve 645, valve 650, and valve 655) are positioned to allow continuous circulation of disinfectant (i.e., 50% H₂O₂ disinfectant) through the disinfection chamber 605 in a closed loop flow. In particular, air flow occurs from the nebulizer 630 to the disinfection chamber 605 and from the disinfection chamber 605, past the turned-off ozone generator 625, to the nebulizer 630. In some examples, during the last 1 minute of the disinfection processing phase, the nebulizer 630 is turned off and the remaining H₂O₂ spray continues to circulate through the system 600.

During the disinfection process, the residual ozone from Phase 1 decreases as H₂O₂ is introduced into the disinfection chamber 605. Although it is known that ozone can be configured to neutralize H₂O₂, the volume of H₂O₂ introduced into the disinfection chamber 605 during the disinfection process is sufficient to overcome these neutralizing effects.

The method 700 can include step 740 - Phase 3 Post-Disinfection Chamber Conditioning - wherein the system 600 clears the disinfection chamber 605 of residual disinfectant. In some embodiments, the post-disinfeciton chamber conditioning of step 740 can last for approximately 2.0 minutes. FIG. 8C illustrates system 600 during the post-disinfection chamber conditioning.

During the post-disinfection chamber conditioning of Phase 3, ozone can be continuously supplied to the disinfection chamber 605 through a closed loop flow. In some embodiments, the residual H₂O₂ micro-spray in the system 600 is neutralized.

A non-limiting example of the status of the components of the system 600 during the Post-Disinfection Chamber Conditioning are provided below:

| | |
|---|---|
| Element | Status |
| circulating fan 610 | ON |
| valve 640 (V1) | OPEN (to internal circulation, closed to exhaust filters) |
| valve 645 (V2) | CLOSED (to inlet filter) |
| valve 655 (V3) | CLOSED (to the chamber) |
| valve 650 (V4) | OPEN (to the nebulizer chamber and internal circulation) |
| pump 660 (Pump 1) | OFF |
| pump 665 (Pump 2) | OFF |
| ozone generator 625 | ON - 2 min |

As illustrated in FIG. 8C, the circulating fan 610 is turned on to circulate air through the system 600. In some examples, the valve 640 is opened to internal circulation within the system 600 but closed to the exhaust filter 615, the valve 645 is closed to the inlet filter, the valve 655 is opened to the nebulizer chamber but closed to the disinfection chamber 605, and the valve 650 is opened to the nebulizer chamber and internal circulation within the system 600. FIG. 8C illustrates that the circulation of air during post-disinfection chamber conditioning allows for ozone to circulate through the nebulizer 630 and disinfection chamber 605. As noted above, this can allow for the ozone to neutralize any remaining H₂O₂. In some examples, the valve 655 can be closed to the nebulizer 630 but opened to the disinfection chamber 605 within the system 600.

In some examples, ozone from the ozone generator 625 is reintroduced into the disinfection chamber 605 for 2 minutes and continuously circulated through the system in a closed loop flow. In some embodiments, as discussed above, the residual H₂O₂ is neutralized by the ozone. After 2 minutes, the ozone generator 625 is turned off.

Method 700 can include step 750 - Phase 4 System Clearing - wherein fresh air is introduced into the system 600 through the inlet filter 620 to flush the disinfection chamber 605. The air can then exit the disinfection chamber 605 and exhausted through the exhaust filter 615. In some embodiments, the inlet filter 620 can be a HEPA filter. In some examples, the exhaust filter 615 can include a HEPA filter and a carbon filter. In some embodiments, the HEPA filters only allow things through less than 0.3 µm particle size. The filtering of the inlet filter 620 and the exhaust filter 615 can ensure that only clean air leaves the system 600 at the end of the method 700. This final phase of the method 700 is configured to provide the system 600 for its next use.

In some embodiments, the Phase 4 System Clearing of step 750 can last for approximatly 1.0 minute. FIG. 8D provides an illustration of the system 600 during the Phase 4 System Clearing.

A non-limiting example of the status of the components of the system 600 during the System Clearing are provided below:

| | |
|---|---|
| Element | Status |
| circulating fan 610 | ON |
| valve 640 (V1) | OPEN (to exhaust filters) |
| valve 645 (V2) | OPEN (from inlet filter to internal circulation) |
| valve 655 (V3) | CLOSED (to the chamber) |
| valve 650 (V4) | OPEN (to nebulizer chamber and internal circulation) |
| pump 660 (Pump 1) | OFF |
| pump 665 (Pump 2) | OFF |
| nebulizer 630 | OFF |
| ozone generator 625 | OFF |

As illustrated in FIG. 8D, the circulating fan 610 is turned on to circulate air through the system 600. In some examples, the valve 640 is opened to the exhaust filter(s) 615, the valve 645 is opened to the inlet filter to allow internal circulation, the valve 655 is opened to the nebulizer chamber but closed to the disinfection chamber 605, and the valve 650 is opened to the nebulizer chamber and internal circulation within the system 600. FIG. 8D illustrates that the circulation of air during system clearing allows for fresh and filtered air to be pulled through the inlet filter 620 to circulate through the nebulizer 630 and the disinfection chamber 605. The air is then exhausted and filtered out of the exhaust filter 615 to ensure that no ozone or H₂O₂ leaves the system. This system clearing phase ensures that the user is not exposed to harmful chemicals.

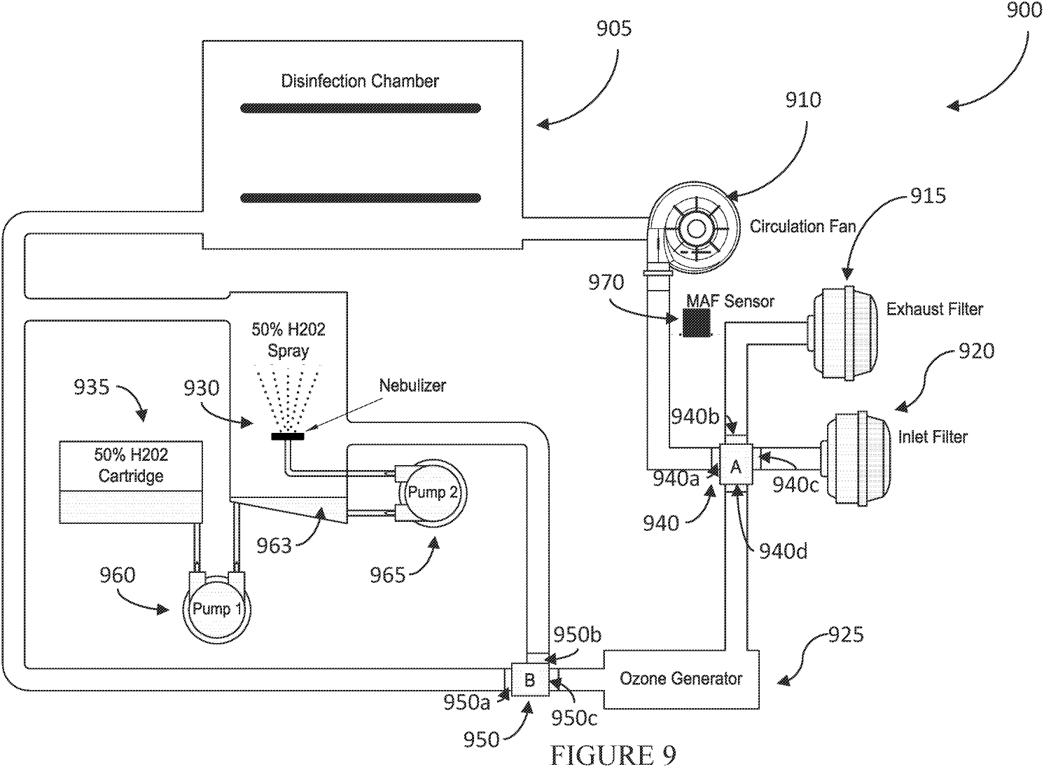

FIG. 9 illustrates a schematic diagram of another embodiment of a system 900 for reducing microorganisms on a surface. The system 900 of FIG. 9 is similar to the system 600 disclosed in FIG. 6 except the number of valves in the system 900 is reduced through the use of 3-way valves. Except where identified, the description of the system 600 of FIGS. 6, 7, 8A-8D applies similarly to the system 900.

As illustrated, the system 900 can be a fully automated and integrated system which can include a chamber 905, an ozone generator 925, a nebulizer 930, at least one pump (i.e., pump 960 or pump 965), a circulating fan 910, a plurality of valves (i.e., valve 940, and valve 950), an inlet and inlet filter 920, and an exhaust filter 915. In some embodiments, the system 900 can include a sensor 970.

In some embodiments, the at least one pump 960, 965 can be a peristaltic pump or other precision pump.

The sensor 970 can be a mass airflow sensor. The sensor 970 can measure the flow rate through the system 900 to ensure consistent flow rate through the system 900 regardless of the path of fluid flow. For example, fluid flow through the nebulizer 930, the pump 960, and the pump 965 can experience a different level of resistance than when when the fluid flow bypasses the the nebulizer 930 and the two pumps 960, 965. The sensor 970 can measure the flow rate and adjust the power delivered to the circulating fan 910 accordingly.

As will be discussed in more detail below, the system 900 can be designed to draw a precise volume between about 1.5 mL and 2.5 mL (e.g., 1.9 mL, 2.0 mL, 2.1 mL, 2.2 mL, 2.3 mL, 2.4 mL, or 2.5 mL) of liquid disinfectant (i.e., 50% H₂O₂) from the cartridge by the at least one pump 960, 965, nebulize the liquid into a spray using the nebulizer 930, and transport the spray via forced air into a continuous closed loop flow through the disinfection chamber 905. In some embodiments, the system 600 can be designed to draw a volume of 1.6 mL, 1.7 mL, 1.8 mL, 1.9 mL, 2.0 mL, 2.1 mL, 2.2 mL, 2.3 mL, 2.4 mL, 2.5 mL, between about 1.6 mL - 1.7 mL, between about 1.7 mL - 1.8 mL, between about 1.8 mL - 1.9 mL, between about 1.9 mL - 2.0 mL, between about 2.0 mL -2.1 mL, between about 2.1 mL - 2.2 mL, between about 2.2 mL - 2.3 mL, between about 2.3 mL - 2.4 mL, between about 2.4 mL - 2.5 mL and any value in between those ranges listed, including endpoints. The disinfectant spray can be contact the surfaces of the items placed in the disinfection chamber 905 to inactivate the pathogens during the disinfectant cycle.

The system 900 can include an ozone generator 925. In some embodiments, the ozone generator 925 can produce ozone. The ozone produced can be used in to ways in the system 900. In some embodiments, the ozone produced by the ozone generator 925 can precondition the disinfection chamber 905. In some examples, the ozone produced by the ozone generator 925 can neutralize the residual H₂O₂ after the item placed in the disinfection chamber 905 has been disinfected.

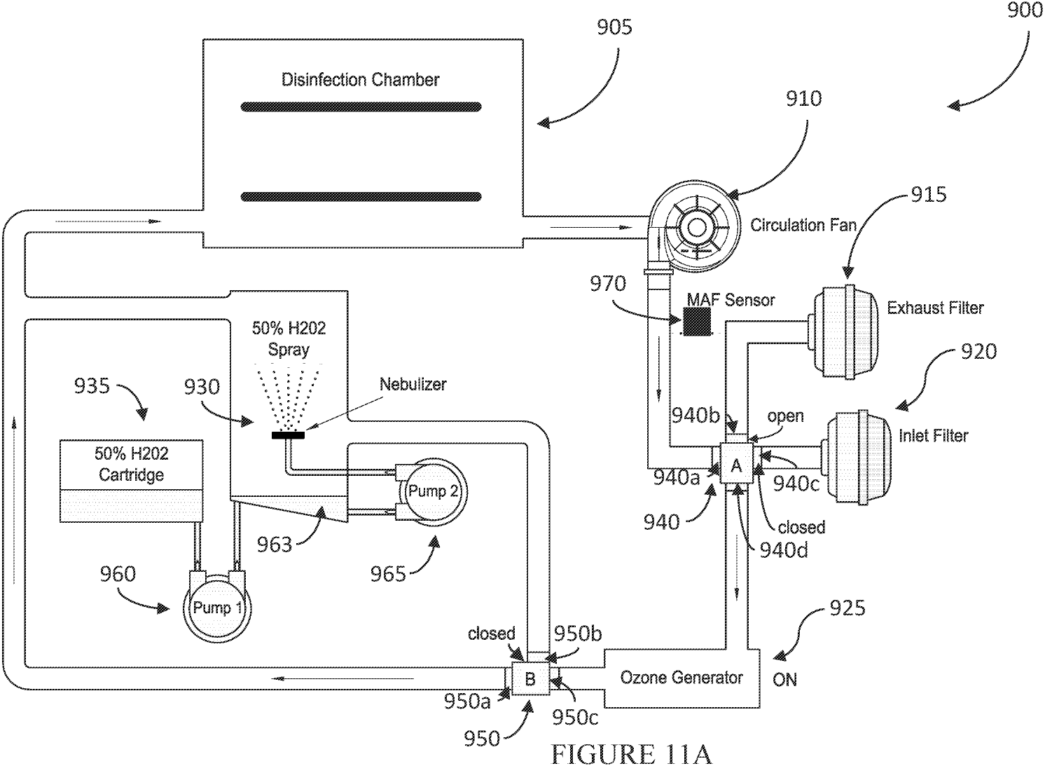

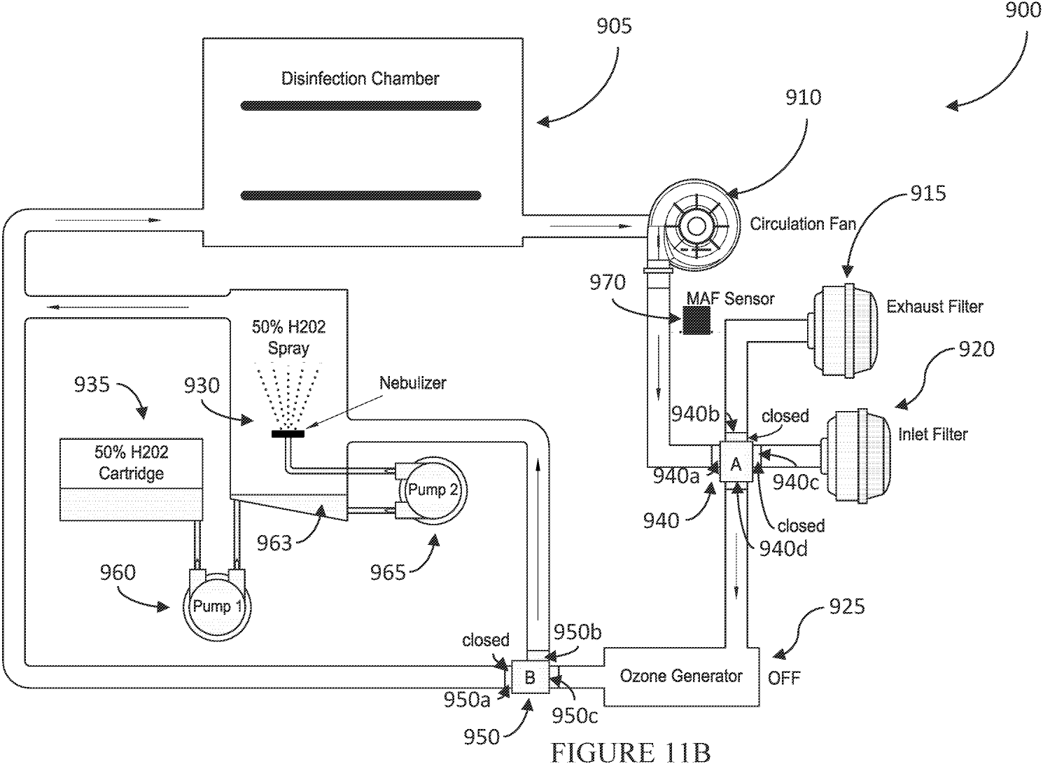

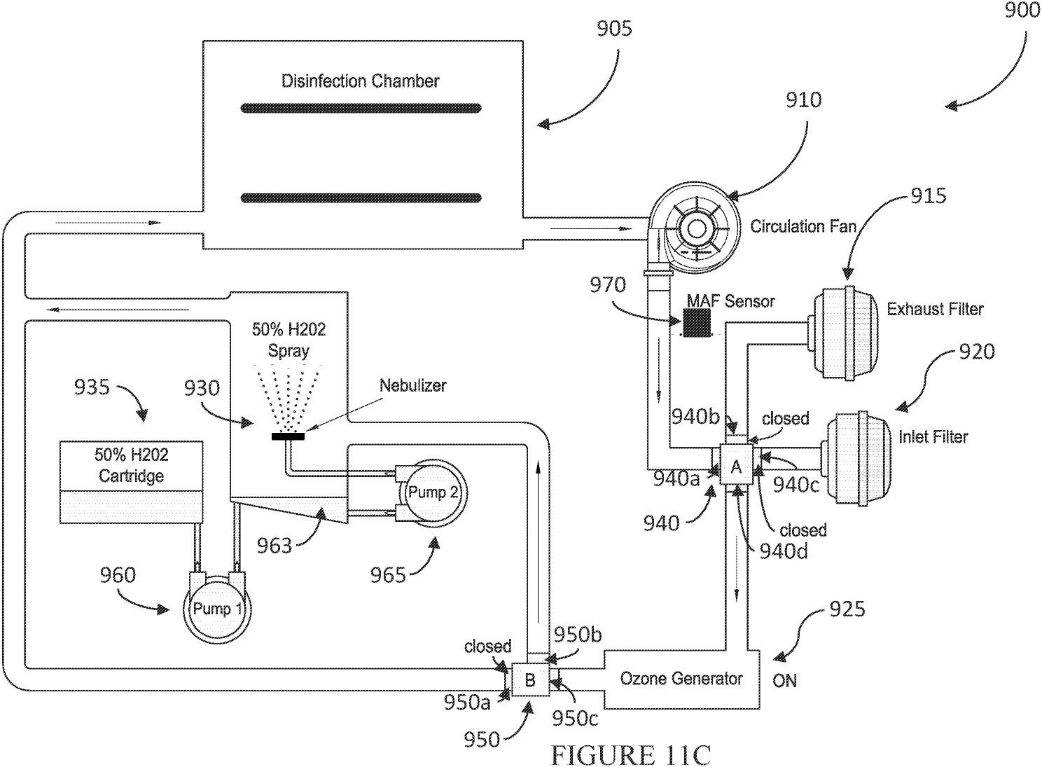

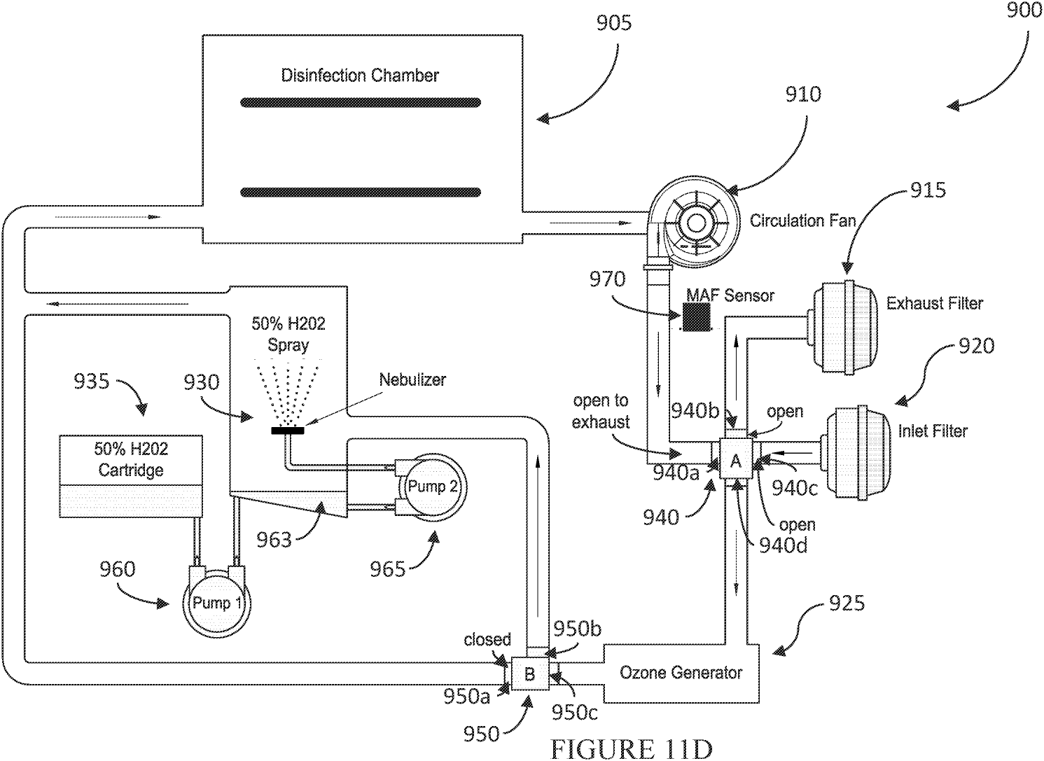

As shown in FIGS. 11A-11D, the system 900 includes a reservoir 963 between the pump 960 and the pump 965. The pump 960 can be preprogrammed to pump out a predetermined amount of disinfectant/sterilant until the cartridge 935 is empty. Because excess disinfectant/sterilant (e.g., H₂O₂) is removed after each disinfection/sterilization cycle, the pump 965 can be responsible for delivering the proper amount of disinfectant/sterilant (e.g., H₂O₂) to the nebulizer 930. Any excess amount of disinfectant/sterilant (e.g., H₂O₂) can be stored in the reservoir 963. In some embodiments, the reservoir 963 has a predetermined amount of disinfectant/sterilant (e.g., H₂O₂) that is stored; if the amount of disinfectant/sterilant (e.g., H₂O₂) falls below the predetermined amount, pump 960 will pump out disinfectant/sterilant to fill the reservoir 963 to the proper amount. In some examples, the volume stored in the reservoir 963 is the amount of disinfectant/sterilant that is delivered to the nebulizer 930. The pump 965 can be programmed to pump whatever is in the reservoir 963. However, if there is any disinfectant/sterilant unused during disinfection/sterilization, the disinfectant/sterilant will be stored in the reservoir 963. When the amount of disinfectant/sterilant of the reservoir 963 falls below the predetermined volume, the pump 660 will pump disinfectant/sterilant out of the cartridge 935 to fill the reservoir 963 to the predetermined volume.

As provided in the table below, the system 900 can be configured to disinfect an item. In some embodiments, the system 900 operates at ambient temperature and ambient pressure conditions in a continuous closed loop flow through the cycle. In some embodiments, the system 900 can operate at a temperature of 20° C., 21° C., 22° C., 23° C., 24° C., 25° C. In some embodiments, the system 900 can operate at a temperature between about 20° C. and 21° C., between about 21° C. and 22° C., between about 22° C. and 23° C., between about 23° C. and 24° C., and between about 24° C. and 25° C. As discussed above, the disclosed system 900 can operate to disinfect without the use of a heater.

In some examples, the system 900 can operate with a relative humidity of 20%, 25%, 30%, 35%, 40%, 45%, 50%, 55%, and 60%. In some embodiments, the system 900 can operate with a relative humidity of between about 20% and 25%, between about 25% and 30%, between about 30% and 35%, between about 35% and 40%, between about 40% and 45%, between about 45% and 50%, between about 50% and 55%, between about 55% and 60%. As discussed above, embodiments of the system 900 do not include a dehumidifier remove moisture from the system 900. The below table provides a summary of an embodiment of the range of operating conditions of the system 900:

| Operating Conditions | Minimum | Maximum |
|---|---|---|
| Temperature | 20° C. | 25° C. |
| Relative Humidity | 20% | 60% |

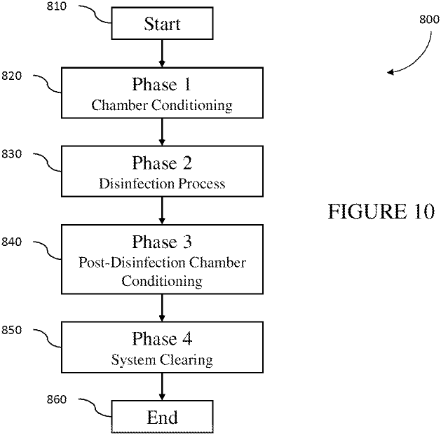

FIG. 10 illustrates a flowchart of a non-limiting method for disinfection 800. As illustrated in the flowchart, the method 800 can start at step 810, proceed to Phase 1 at step 820, proceed to Phase 2 at step 830, proceed to Phase 3 at step 840, proceed to Phase 4 at step 850, and end at step 860. Each of these phases will be discussed in more detail below. In some embodiments, the process time for the method 800 is 10 minutes and includes four distinct phases. In some examples, the contact time with the disinfectant (i.e., H₂O₂) is approximately 4.5 minutes of the 10-minute process. A summary of the time for each phase of an embodiment of method 700 is provided below:

| Phase | Description | Phase Duration (min) | Elapsed Time (min) |
|---|---|---|---|
| Phase 1 | Chamber Conditioning | 2.5 | 0 - 2.5 |
| Phase 2 | Disinfection Process (contact time) | 4.5 | 2.5 - 7.0 |
| Phase 3 | Post-Disinfection Chamber Conditioning | 2.0 | 7.0 - 9.0 |
| Phase 4 | System Clearing | 1.0 | 9.0 - 10.0 |

In some embodiments, the Phase 1 Chamber Conditioning phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 - 1.0 minutes, between about 1.0 - 1.5 minutes, between about 1.5 - 2.0 minutes, between about 2.0 - 2.5 minutes, between about 2.5 - 3.0 minutes, between about 3.0 - 3.5 minutes, between about 3.5 - 4.0 minutes, between about 4.0 - 4.5 minutes, between about 4.5 - 5.0 minutes and any value in between those ranges listed, including endpoints. In some embodiments, the Phase 2 Disinfection Process phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 - 1.0 minutes, between about 1.0 - 1.5 minutes, between about 1.5 - 2.0 minutes, between about 2.0 - 2.5 minutes, between about 2.5 - 3.0 minutes, between about 3.0 - 3.5 minutes, between about 3.5 - 4.0 minutes, between about 4.0 - 4.5 minutes, between about 4.5 - 5.0 minutes and any value in between those ranges listed, including endpoints. In some embodiments, the Phase 3 Post-Disinfection Chamber Conditioning phase phase can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 - 1.0 minutes, between about 1.0 - 1.5 minutes, between about 1.5 - 2.0 minutes, between about 2.0 - 2.5 minutes, between about 2.5 - 3.0 minutes, between about 3.0 - 3.5 minutes, between about 3.5 - 4.0 minutes, between about 4.0 - 4.5 minutes, between about 4.5 - 5.0 minutes and any value in between those ranges listed, including endpoints. In some examples, the Phase 4 System Clearing can have a duration of 0.5 minutes, 1.0 minutes, 1.5 minutes, 2.0 minutes, 2.5 minutes, 3.0 minutes, 3.5 minutes, 4.0 minutes, 4.5 minutes, 5.0 minutes, between about 0.5 - 1.0 minutes, between about 1.0 -1.5 minutes, between about 1.5 - 2.0 minutes, between about 2.0 - 2.5 minutes, between about 2.5 - 3.0 minutes, between about 3.0 - 3.5 minutes, between about 3.5 - 4.0 minutes, between about 4.0 - 4.5 minutes, between about 4.5 - 5.0 minutes and any value in between those ranges listed, including endpoints.

In some embodiments, the method 800 can start at step 810. Prior to inserting the item(s) to be disinfected in the disinfection chamber 905, the user must first clean and dry the items to be placed in the disinfection chamber 905. Once the item is clean and dry, the user can place the disinfection chamber 905.

The method 800 can include step 820 - Phase 1 Chamber Conditioning - wherein the disinfection chamber 905 is conditioned. In some embodiments, the chamber conditioning step of step 820 can last for approximately 2.5 minutes. FIG. 11A provides an illustration of the method 800 during the chamber conditioning.

As the name suggests, during the chamber conditioning of Phase 1, the ozone generator 925 conditions the chamber for H₂O₂ disinfection by converting H₂O to OH radicals, thereby reducing residual moisture. In some embodiments, during the chamber conditioning phase of step 820, ozone is supplied by the ozone generator 925 to the disinfection chamber 905 through a closed loop flow. Phase 1 Chamber Conditioning can optimize the disinfection chamber 905 for disinfection. In some examples, the disinfection chamber 905 is optimized for H₂O₂ disinfection.

A non-limiting example of the status of the components of the system 900 during Phase 1 Chamber Conditioning of step 820 is provided below:

| | |
|---|---|
| Element | Status |
| circulating fan 910 | ON |
| opening 940a of valve 940 | OPEN (to internal circulation) |
| opening 940b of valve 940 | OPEN (to exhaust filter) |
| opening 940c of valve 940 | CLOSED (to inlet filter) |
| opening 940d of valve 940 | OPEN (to internal circulation) |
| opening 950a of valve 950 | OPEN (to internal circulation) |
| opening 950b of valve 950 | CLOSED |
| opening 950c of valve 950 | OPEN (to internal circulation) |
| pump 960 (Pump 1) | ON (fills the nebulizer reservoir) |
| pump 965 (Pump 2) | OFF |
| ozone generator 925 | ON - first 1.5 min of 2.5 min phase |
| | OFF - last 1.0 min of 2.5 min phase |

As illustrated in FIG. 11A, the circulating fan 910 is turned on to circulate air through the system 900. The opening 940a and opening 940b of the valve 940 and opening 950a and opening 950c of the valve 950 can be opened to allow internal circulation of ozone. In some examples, the pump 960 is “ON” which allows the nebulizer reservoir to fill. During Phase 1, the pump 965 can be turned “OFF” as the chamber conditioning does not use any H₂O₂.

In some examples, the ozone generator 925 is turned on for part of the chamber conditioning phase of step 820. The ozone generator 925 can be turned on for the first part of the chamber conditioning phase. For example, this can be the first 1.5 minutes of the 2.5 minute chamber conditioning phase. This can allow ozone to be supplied to the disinfection chamber 905 from the ozone generator 925 for a duration of time. As shown in FIG. 11A, opening 940a and opening 940d of the valve 940 and opening 950a and opening 950c of the valve 950 are opened to allow continuous circulation of ozone through the disinfection chamber 905 in a closed loop flow. In particular, air flow occurs from the ozone generator 925 to the disinfection chamber 905 and from the disinfection chamber 905 to the ozone generator 925. The circulation of ozone during this phase conditions the chamber for H₂O₂ disinfection by converting H₂O molecules to OH radicals (disinfecting molecules) and thereby reducing residual moisture.

The ozone generator 925 can be turned off for a second part of the chamber conditioining phase. In some embodiments, the ozone generator 925 is turned off for the last 1.0 minute of the 2.5 minute chamber conditionining phase. During the second part of the chamber conditioning phase (i.e., the last 1.0 minute of the 2.5 minute phase), when the ozone generator 925 is turned off, the ozone level will decay over time as it interacts with surfaces within the system 900. In some embodiments, the sensor 970 can achieve equilibrium with the outside pressure through the exhaust filter 915. As shown, the opening 940b of the valve 940 can remain open to ensure that no vacuum is created within the system 900.

Method 800 can include step 830 - Phase 2 Disinfection Process -wherein an item placed in the disinfection chamber 905 is disinfected. In some examples, the Phase 2 Disinfection Process of step 830 can last for approximately 4.5 minutes. FIG. 11B provides an illustration of the system 900 during the Phase 2 Disinfection Process.

During the Phase 2 Disinfection Process, the disinfectant is introduced into the disinfection chamber 905. In some embodiments, the disinfectant is a 50% hydrogen peroxide solution. The disinfectant can be introduced into the 905 through the nebulizer 930. The nebulizer 930 can convert the disinfectant (i.e., the 50% hydrogen peroxide solution) from a liquid into a micro-spray that allows the disinfectant to move in the closed loop flow. In some embodiments, the micro-spray is the active ingredient used in the disinfection process.

A non-limiting example of the status of the components of the system 900 during the Disinfection Process is provided below.

| | |
|---|---|
| Element | Status |
| circulating fan 910 | ON |
| opening 940a of valve 940 | OPEN (to internal circulation) |
| opening 940b of valve 940 | OPEN (to exhaust filter) |
| opening 940c of valve 940 | CLOSED (to inlet filter) |
| opening 940d of valve 940 | OPEN (to internal circulation) |
| opening 950a of valve 950 | CLOSED (to the chamber) |
| opening 950b of valve 950 | OPEN (to nebulizer and internal circulation) |
| opening 950c of valve 950 | OPEN (to nebulizer and internal circulation) |
| pump 960 (Pump 1) | OFF |
| pump 965 & nebulizer 930 (Pump 2 & Nebulizer) | ON (delivers 2.1 mL and nebulizes for 3.5 min.) |
| ozone generator 925 | OFF |

As illustrated in FIG. 11B, the circulating fan 910 is turned on to circulate air through the system 900. In some examples, the opening 940a and opening 940b of the valve 940 are opened while the opening 940c is closed. In some embodiments, the opening 950b and opening 950c of the valve 950 are opened while the opening 950a is closed. The pump 960 is turned “OFF” to prevent the disinfectant (e.g., 50% H₂O₂ solution) from filling the nebulizer 930. In some examples, the pump 965 and the nebulizer 930 are turned “ON” to deliver disinfectant through the nebulizer 930.

In some embodiments, 50% H₂O₂ is the active ingredient in the disinfection process of nebulizer 930. During the disinfection process, the pump 965 is a peristaltic pump that is fluidly connected to the nebulizer 930. In some embodiments, the nebulizer 930 is an 8-micron nebulizer mesh. The pump 965 can be configured to deliver approximaly 2.1 mL of 50% H₂O₂ disinfectant to the nebulizer 630 for the first 3.5 minutes of the contact time. In some embodiments, the system 600 can be designed to deliver a volume of 1.6 mL, 1.7 mL, 1.8 mL, 1.9 mL, 2.0 mL, 2.1 mL, 2.2 mL, 2.3 mL, 2.4 mL, 2.5 mL, between about 1.6 mL - 1.7 mL, between about 1.7 mL - 1.8 mL, between about 1.8 mL - 1.9 mL, between about 1.9 mL - 2.0 mL, between about 2.0 mL - 2.1 mL, between about 2.1 mL - 2.2 mL, between about 2.2 mL - 2.3 mL, between about 2.3 mL - 2.4 mL, between about 2.4 mL - 2.5 mL and any value in between those ranges listed, including endpoints. In some embodiments, the pump 965 is programed to deliver 0.01 mL of 50% H₂O₂ per second for 3.5 minutes (210 seconds) to the nebulizer 930. The H₂O₂ solution can then be nebulized into a spray and be continuously circulated through the disinfection chamber 905 in a closed flow loop. As shown in FIG. 11B, the openings of each of the valve 940 and the valve 950 are positioned to allow continuous circulation of disinfectant (i.e., 50% H₂O₂ disinfectant) through the disinfection chamber 905 in a closed loop flow. In particular, air flow occurs from the nebulizer 930 to the disinfection chamber 905 and from the disinfection chamber 905 disinfection chamber 905, past the turned off ozone generator 925, to the nebulizer 930. In some examples, during the last minute of the disinfection processing phase, the nebulizer 930 is turned off and the remaining H₂O₂ spray continues to circulate through the system 900.

In some examples, during the disinfection process of step 830, the residual ozone from Phase 1 of step 820 decreases as H₂O₂ is introduced into the disinfection chamber 905. Although it is known that ozone can be configured to neutralize H₂O₂, the volume of H₂O₂ introduced into the disinfection chamber 905 during the disinfection process is sufficient to overcome those neutralizing effects. In some embodiments, the sensor 970 can achieve equilibrium with the outside pressure through the exhaust filter 915. As shown, the opening 940b of the valve 940 can remain open to ensure that no vacuum is created within the system 900.

The method 800 can include step 840 - Phase 3 Post-Disinfection Chamber Conditioning - wherein the system 900 clears the disinfection chamber 905 of residual disinfectant. In some embodiments, the post-disinfection chamber conditioning of step 840 can last for approximately 2.0 minutes. FIG. 11C illustrates system 900 during the post-disinfection chamber conditioning.

During the post-disinfection chamber conditioning of Phase 3, ozone can be continuously supplied to the disinfection chamber 905 through a closed loop flow. In some embodiments, the residual H₂O₂micro-spray in the system 600 is neutralized.

A non-limiting example of the status of the components of the system 900 during the Post-Disinfection Chamber Conditioning are provided below:

| | |
|---|---|
| Element | Status |
| circulating fan 910 | ON |
| opening 940a of valve 940 | OPEN (to internal circulation) |
| opening 940b of valve 940 | OPEN (to exhaust filter) |
| opening 940c of valve 940 | CLOSED (to inlet filter) |
| opening 940d of valve 940 | OPEN (to internal circulation) |
| opening 950a of valve 950 | CLOSED (to the chamber) |
| opening 950b of valve 950 | OPEN (to nebulizer and internal circulation) |
| opening 950c of valve 950 | OPEN (to nebulizer and internal circulation) |
| pump 960 (Pump 1) | OFF |
| pump 965 (Pump 2) | OFF |
| ozone generator 925 | ON - 2 min |

As illustrated in FIG. 11C, the circulating fan 910 is turned on to circulate air through the system 900. In some examples, the valve 940 is opened to internal circulation within the system 900 but closed to the exhaust filter 915 and the inlet filter 920. In some embodiments, the valve 950 is closed to the disinfection chamber 905 but opened to the nebulizer 930 and internal circulation within the system 900. FIG. 11C illustrates that the circulation of air during post-disinfection chamber conditioning allows for ozone to circulate through the nebulizer 930 and the disinfection chamber 905. As noted above, this can allow for the ozone to neutralize any remaining H₂O₂. In some examples, the valve 950 can be closed to the nebulizer 930 but opened to the disinfection chamber 905 within the system 900.

During the Post-Disinfection Chamber Conditioining phase of 840, ozone from the ozone generator 925 is reintroduced into the disinfection chamber 905 for 2 minutes and continuously circulated through the system 900 in a closed loop flow. In some embodiments, as discussed previously, the residual H₂O₂ is neutralized by the ozone. After 2 minutes, the ozone generator 925 is turned off. In some embodiments, the sensor 970 can achieve equilibrium with the outside pressure through the exhaust filter 915. As shown, the opening 940b of the valve 940 can remain open to ensure that no vacuum is created within the system 900.

The method 800 can include step 850 - Phase 4 System Clearing -wherein fresh air is introduced into the system 900 through the inlet filter 920 to flush and purge the disinfection chamber 905. The air can then exit the disinfection chamber 905 and is exhausted through the exhaust filter 915. In some embodiments, the inlet filter 920 can be a HEPA filter. In some examples, the exhaust filter 915 can include a HEPA filter and a carbon filter. In some embodiments the HEPA filters only allow things less than 0.3 µm particle size through the filter. The filtering of the inlet filter 920 and the exhaust filter 915 can ensure that only clean air leaves the system 900 at the end of the method 800. This final phase of the method 800 can prepare the system 900 for its subsequent use.

In some embodiments, the System Clearing phase of step 850 can last for approximately 1.0 minute. FIG. 11D provides an illustration of the system 900 during the Phase 4 System Clearing.

A non-limiting example of the status of the components of the system 900 during System Clearing are provided below:

| | |
|---|---|
| Element | Status |
| circulating fan 910 | ON |
| opening 940a of valve 940 | OPEN (to exhaust filter only) |
| opening 940b of valve 940 | OPEN (to exhaust filter) |
| opening 940c of valve 940 | OPEN (to internal circulation) |
| opening 940d of valve 940 | OPEN (to internal circulation) |
| opening 950a of valve 950 | CLOSED (to the chamber) |
| opening 950b of valve 950 | OPEN (to nebulizer and internal circulation) |
| opening 950c of valve 950 | OPEN (to nebulizer and internal circulation) |
| pump 960 (Pump 1) | OFF |
| pump 965 (Pump 2) | OFF |
| ozone generator 925 | OFF |

As illustrated in FIG. 11D, the circulating fan 910 is turned on to circulate air through the system 900. In some examples, the openings of the valve 940 are opened to allow air flow from the internal circulation out of the exhaust filter and for air flow into the internal circulation from the internal filter. The opening 950b and opening 950c of the valve 950 are opened to allow airflow through the nebulizer chamber and internal circulation while opening 950a is closed to the disinfection chamber 905. As shown in FIG. 11D, the valve 940 and valve 950 provides for the circulation of air during system clearing to allow for fresh and filtered air to be pulled through the inlet filter 920 and to circulate through the nebulizer 930 and the disinfection chamber 905. The air is then exhausted and filtered out of the exhaust filter 915 to ensure that no ozone or H₂O₂ leaves the system. This system clearing phase ensures that the user is not exposed to harmful chemicals.

### User Engagement With the Method for Disinfection, Sterilization, and/or Sanitization

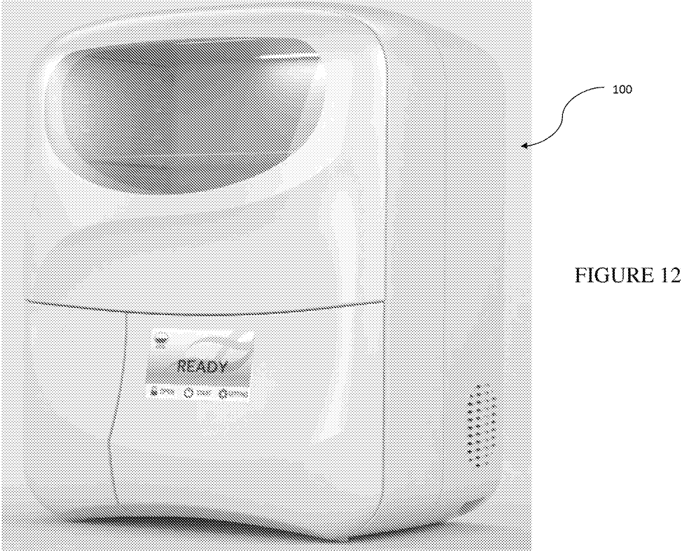

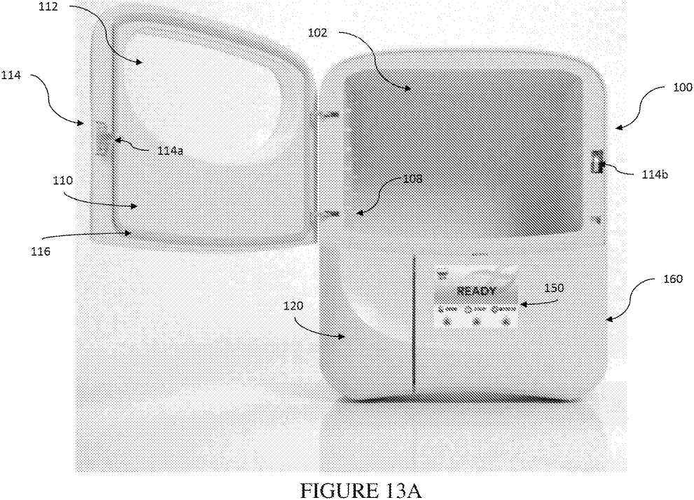

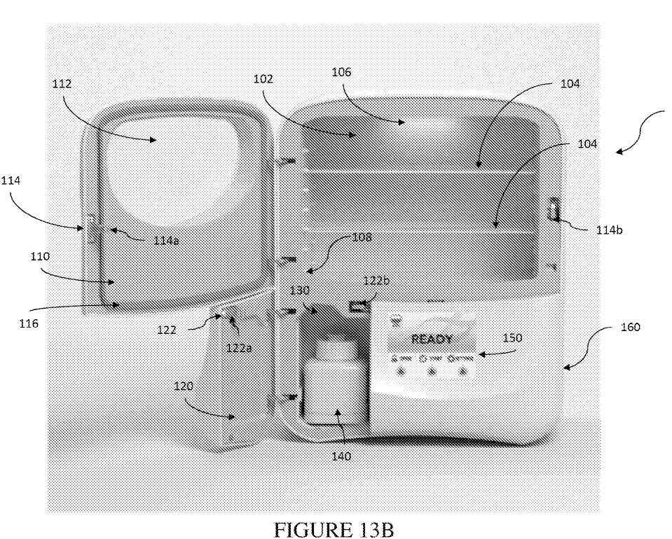

FIGS. 12-19 illustrate the user engagement with the system 100 described above during the method 700. FIG. 12 illustrates the system 100 as it is ready to disinfect one or more items. As shown in FIGS. 13A-13B, the user can open the chamber door 110 to access chamber 102 of the system 100. The chamber 102 can include a plurality of pegs 108 where the user can place a number of adjustable shelves 104 depending on the size and number of items 101 to be disinfected. In some embodiments, prior to beginning the method 700, the user can open the cartridge door 120 to replace the cartridge 140.

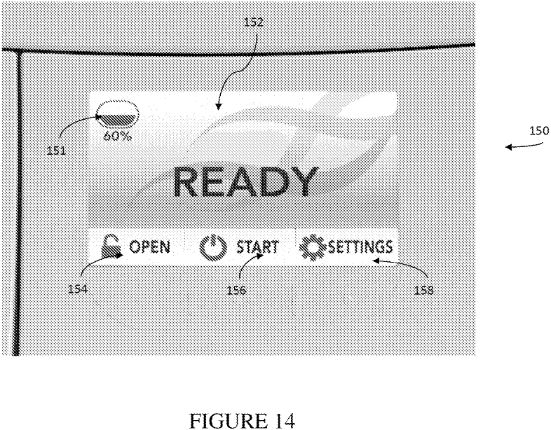

As discussed above, the system 100 can include a screen 150 that allows a user to control the operation of the system 100 (implementation mechanisms are dicsussed in more detail below). In some embodiments, the screen 150 is configured to allow a user to activate the method 700 in the system 100. FIG. 14 illustrates an enlarged view of the screen 150. In some embodiments, the screen 150 can include a display 152, a plurality of buttons (e.g., button 154, button 156, and button 158), and at least one indicator 151. In some embodiments, the display 152 displays information about the status of the system 100 or the status of the progress of the method 700 as the items 101 are being disinfected in the system 100. The screen 150 can include a button 154, a button 156, and a button 158. In the embodiment illustrated in FIG. 14, the button 154 is configured to either engage or disengage the chamber door 110 of the system 100 to open the chamber door 110. As shown in FIG. 14, the button 156 can allow a user to start and/or stop the progression of the disinfecting the at least one items 101 in the system 100. In some embodiments, the button 158 is configured to allow the user to change and/or review the settings of the system 100. In some embodiments, the screen 150 can include the indicator 151 that provides the user with information regarding the system 100. For example, as illustrated in FIG. 14, the indicator 151 can provide information regarding the amount of disinfectant remaining in the cartridge 140. In some embodiments, the indicator 151 can be configured to indicate that an error has occurred in the system 100. FIG. 14 illustrates an embodiment of the screen 150 when the system 100 is prepared to receive at least one items 101 for disinfecting.

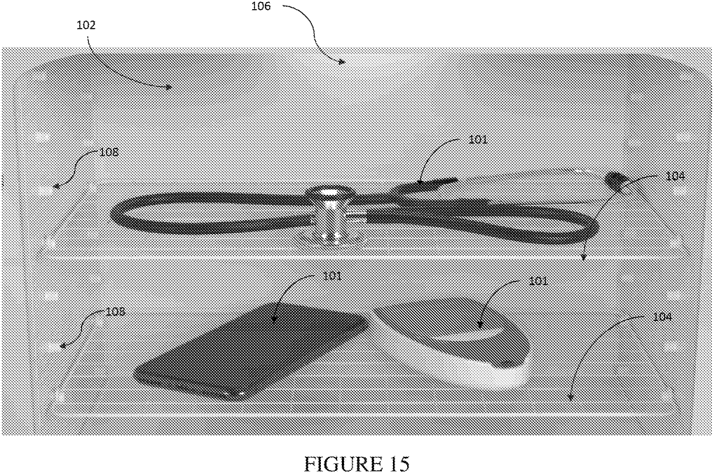

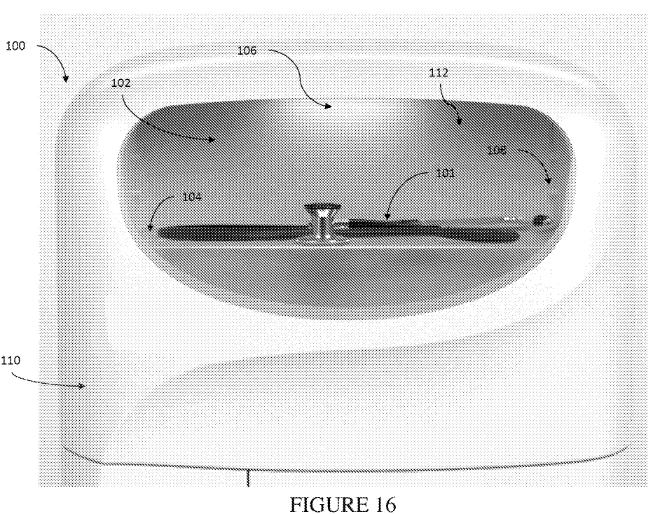

FIG. 15 illustrates an enlarged view of the chamber 102 an example of the positioning of two adjustable shelves 104 and the positioning of a plurality of items 101 on the adjustable shelves 104 for disinfecting. FIG. 16 illustrates a view of system 100 when the chamber door 110 is closed. As shown, the viewing window 112 allows a user to view the the items 101 as they are being disinfected. As well, the light 106 illuminates the inside of the viewing window 112 and can also be seen by the user through the viewing window 112. In some embodiments, before the method for disinfecting starts (i.e., method 700), the light 106 can have a first color that indicates to the user that disinfecting of the items 101 has not started.

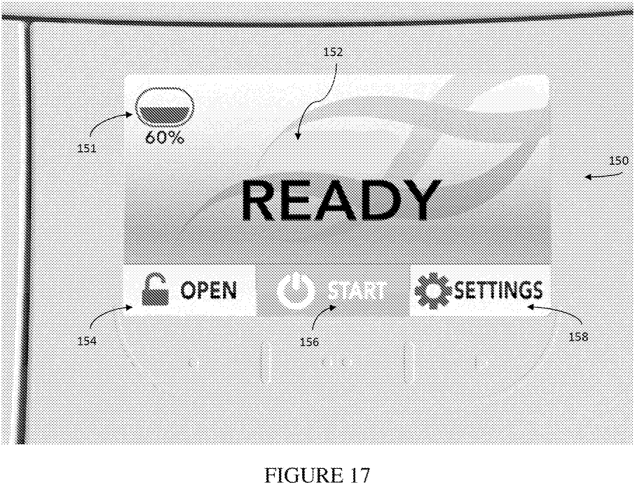

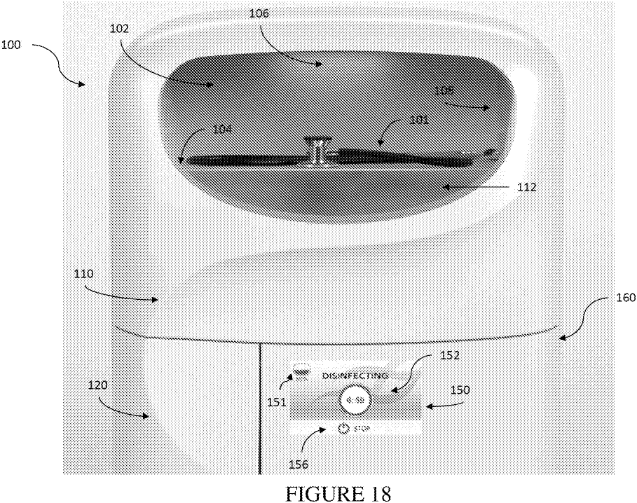

As shown in FIG. 17, to start the disinfecting process, the user can activate the button 156. In some embodiments, when the user engages one of the buttons, the engaged button can change visually. FIG. 18 illustrates an embodiment of the system 100 when the at least one items 101 are being disinfected. As discussed above, the screen 150 includes information on the display 152 that lets the user know the status of the system 100. For example, the display 152 tells the user that the at least one items 101 are “disinfecting” and the time remaining for that process. The display 152 can also change to include a single button - button 156 - that allows the user to stop the disinfecting process. In some embodiments, the light 106 can have a second color that indicates to the user that the at least one items 101 are being disinfected.

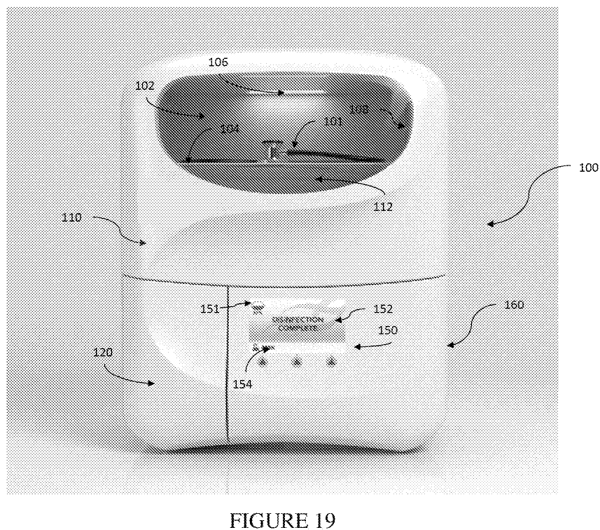

FIG. 19 illustrates an embodiment of the system 100 once the at least one items 101 are disinfected. As shown, the screen 150 can include information on the display 152 that lets the user know the status of the system 100. In FIG. 19,the display 152 indicates to the user that the disinfection is complete and provides the user with a button - button 154 - that allows the user to open the chamber door 110. In some embodiments, the light 106 can have a third color that indicates to the user that the at least one items 101 are done being disinfected and the chamber door 110 can be safely opened.

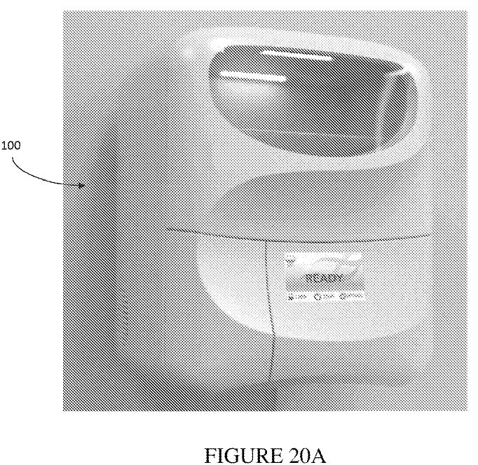

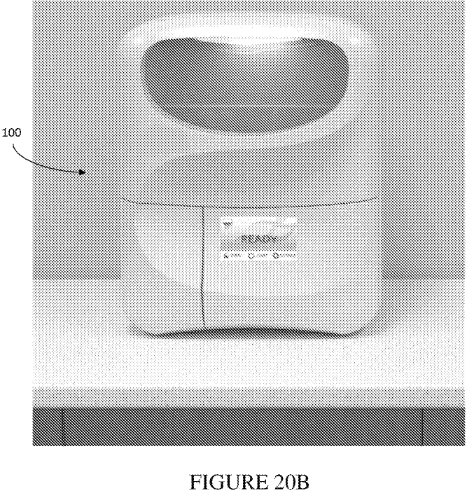

FIGS. 20A-20B illustrates non-limitng embodiments of placement of the system 100 in an environment. As illustrated, the system 100 can have a compact design that can easily fit into a variety of environments. As illustrated in FIG. 20A, the system 100 can be wall mounted. As illustrated in FIG. 20B, the system 100 can be placed on a countertop. In some embodiments, the design of the system 100 only requires a standard outlet and no venting is required.

### Implementation Mechanisms

According to some embodiments, the methods described herein can be implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, server computer systems, portable computer systems, handheld devices, networking devices or any other device or combination of devices that incorporate hard-wired and/or program logic to implement the techniques.

Computing device(s) are generally controlled and coordinated by operating system software, such as iOS, Android, Chrome OS, Windows XP, Windows Vista, Windows 7, Windows 8, Windows Server, Windows CE, UNIX, Linux, SunOS, Solaris, iOS, Blackberry OS, VxWorks, or other compatible operating systems. In other embodiments, the computing device may be controlled by a proprietary operating system. Conventional operating systems control and schedule computer processes for execution, perform memory management, provide file system, networking, I/O services, and provide a user interface functionality, such as a graphical user interface (“GUI”), among other things.

In some embodiments, the computer system includes a bus or other communication mechanism for communicating information, and a hardware processor, or multiple processors, coupled with the bus for processing information. Hardware processor(s) may be, for example, one or more general purpose microprocessors.

In some embodiments, the computer system may also include a main memory, such as a random access memory (RAM), cache and/or other dynamic storage devices, coupled to a bus for storing information and instructions to be executed by a processor. Main memory also may be used for storing temporary variables or other intermediate information during execution of instructions to be executed by the processor. Such instructions, when stored in storage media accessible to the processor, render the computer system into a special-purpose machine that is customized to perform the operations specified in the instructions.

In some embodiments, the computer system further includes a read only memory (ROM) or other static storage device coupled to bus for storing static information and instructions for the processor. A storage device, such as a magnetic disk, optical disk, or USB thumb drive (Flash drive), etc., may be provided and coupled to the bus for storing information and instructions.

In some embodiments, the computer system may be coupled via a bus to a display, such as a cathode ray tube (CRT) or LCD display (or touch screen), for displaying information to a computer user. An input device, including alphanumeric and other keys, is coupled to the bus for communicating information and command selections to the processor. Another type of user input device is cursor control, such as a mouse, a trackball, or cursor direction keys for communicating direction information and command selections to the processor and for controlling cursor movement on display. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

In some embodiments, the computing system may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage

In some embodiments, a computer system may implement the methods described herein using customized hard-wired logic, one or more ASICs or FPGAs, firmware and/or program logic which in combination with the computer system causes or programs the computer system to be a special-purpose machine. According to one embodiment, the methods herein are performed by the computer system in response to hardware processor(s) executing one or more sequences of one or more instructions contained in main memory. Such instructions may be read into main memory from another storage medium, such as a storage device. Execution of the sequences of instructions contained in main memory causes processor(s) to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, or other types of storage devices. Volatile media includes dynamic memory, such as a main memory. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between nontransitory media. For example, transmission media includes coaxial cables, copper wire and fiber optics, including the wires that comprise a bus. Transmission media can also take the form of acoustic or light waves, such as those generated during radiowave and infra-red data communications.

Various forms of media may be involved in carrying one or more sequences of one or more instructions to a processor for execution. For example, the instructions may initially be carried on a magnetic disk or solid state drive of a remote computer. The remote computer can load the instructions into its dynamic memory and send the instructions over a telephone line using a modem or other network interface, such as a WAN or LAN interface. A modem local to a computer system can receive the data on the telephone line and use an infra-red transmitter to convert the data to an infra-red signal. An infra-red detector can receive the data carried in the infra-red signal and appropriate circuitry can place the data on a bus. The bus carries the data to the main memory, from which the processor retrieves and executes the instructions. The instructions received by the main memory may retrieve and execute the instructions. The instructions received by the main memory may optionally be stored on a storage device either before or after execution by the processor.

In some embodiments, the computer system may also include a communication interface coupled to a bus. The communication interface may provide a two-way data communication coupling to a network link that is connected to a local network. For example, a communication interface may be an integrated services digital network (ISDN) card, cable modem, satellite modem, or a modem to provide a data communication connection to a corresponding type of telephone line. As another example, a communication interface may be a local area network (LAN) card to provide a data communication connection to a compatible LAN (or WAN component to communicate with a WAN). Wireless links may also be implemented. In any such implementation, a communication interface sends and receives electrical, electromagnetic or optical signals that carry digital data streams representing various types of information.

A network link may typically provide data communication through one or more networks to other data devices. For example, a network link may provide a connection through a local network to a host computer or to data equipment operated by an Internet Service Provider (ISP). The ISP in turn provides data communication services through the world wide packet data communication network now commonly referred to as the “Internet.” The local network and Internet both use electrical, electromagnetic or optical signals that carry digital data streams. The signals through the various networks and the signals on the network link and through a communication interface, which carry the digital data to and from the computer system, are example forms of transmission media.

In some embodiments, the computer system can send messages and receive data, including program code, through the network(s), the network link, and the communication interface. In the Internet example, a server might transmit a requested code for an application program through the Internet, ISP, local network, and communication interface.

The received code may be executed by a processor as it is received, and/or stored in a storage device, or other non-volatile storage for later execution.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, subranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

What is claimed is:

1. A system for reducing the viability of microorganisms on a surface, comprising:
a chamber configured to receive an item to be disinfected, sterilized, or sanitized;
a cartridge configured to contain a solution comprising hydrogen peroxide;
a reservoir configured to receive excess hydrogen peroxide solution;
a nebulizer configured to convert hydrogen peroxide into a vapor
a first peristaltic pump fluidly connected to the cartridge and the reservoir;
a second peristaltic pump fluidly connected to the reservoir and the nebulizer, wherein the second peristaltic pump is configured to deliver hydrogen peroxide from the reservoir to the nebulizer;
an ozone generator configured to generate ozone;
a fan configured to circulate air, including ozone, through the system;
an inlet configured to allow air to flow into the system;
an outlet configured to allow air to flow out of the system;
a first valve configured to control fluid flow into and out of the system; and
a second valve configured to control fluid flow to the nebulizer.

2. The system of claim 1, wherein the system does not include a heater.

3. The system of claim 1, wherein the system does not include a humidifier or a dehumidifier.

4. The system of claim 1, wherein the system does not include a desiccator.

5. The system of claim 1, wherein the blower maintains the system at a slight negative pressure.

6. The system of claims 1, wherein the system further includes a fluid flow sensor intended to maintain constant fluid flow in the system.

7. The system of claims 1, wherein the system is configured to operate at a temperature between 20° C. to 25° C.

8. The system of claim 1, wherein the system is configured to operate with a relative humidity between 20% and 60%.

9. The system of claims 1, wherein the second peristaltic pump is configured to deliver a predetermined quantity of hydrogen peroxide solution to the nebulizer.

10. The system of claims 1 wherein the first peristaltic pump is configured to maintain a predetermined quantity of hydrogen peroxide solution in the reservoir.

11. A method for reducing viable microbial burden on a surface comprising:
placing at least one item into a chamber of a system according to any one of claims 1-10;
activating a conditioning phase to circulate ozone in the system;
activating a disinfection phase wherein the hydrogen peroxide solution is nebulized and is circulated through the system;
activating a post-disinfection conditioning phase to circulate ozone in the system; and
activating a system clearing phase to pull air into the system through the inlet and exhaust the air out of the outlet.

12. A method for reducing viable microbial burden on a surface, the method comprising:
placing at least one item into a chamber of a system for reducing microorganism viability, wherein the system comprises a nebulizer configured to convert hydrogen peroxide solution into a vapor, a cartridge configured to contain the hydrogen peroxide solution, at least one peristaltic pump, an ozone generator, a blower, an inlet and an outlet;
activating a conditioning phase to circulate ozone from the ozone generator in the system, wherein the ozone is configured to convert H₂O molecules to OH radicals so as to reduce residual moisture in the system;
activating a disinfection phase wherein the hydrogen peroxide solution is nebulized into a spray and is circulated through the system;
activating a post-disinfection conditioning phase to circulate ozone from the ozone generator in the system, wherein the ozone is configured to neutralize any remaining H₂O₂ in the system; and
activating a system clearing phase to pull air into the system through the inlet, circulate the air through the nebulizer and the chamber, and exhaust the air out of the outlet.

13. The method of claim 12, wherein the disinfection phase operates at a temperature between 20° C. to 25° C.

14. The method of claim 12, wherein the system operates with a relative humidity between 20% and 60%.

15. The method of any of claims 12-14, wherein the conditioning phase has a duration of at least 2.5 minutes.

16. The method of claim 15, wherein the disinfection phase has a duration of at least 4.5 minutes.

17. The method of claim 16, wherein the post-disinfection phase has a duration of at least 2 minutes.

18. The method of claim 17, wherein the system clearing phase has a duration of at least 1 minute.

19. The method of claim 12, wherein the system does not include a heater configured to dry the system.

20. The method of claim 12, wherein the system does not include a humidifier or a dehumidifier.

21. The method of claim 12, wherein the system does not include a desiccator.

22. The method of any of claims 12-21, wherein fluid flow during the conditioning phase circulates fluid flow that bypasses the nebulizer.

23. The method of claim 22, wherein fluid flow during the disinfection phase circulates fluid flow through the nebulizer.

24. The method of claim 23, wherein fluid flow during the post-disinfection conditioning phase circulates fluid flow through the nebulizer.

25. The method of claim 24, wherein fluid flow during the clearing phase circulates fluid flow that bypasses the nebulizer.

26. A system for reducing the viability of microorganisms on a surface, comprising:
a chamber configured to contain an item to be sterilized, disinfected, sanitized, or decontaminated;
a reservoir configured to contain a disinfectant;
a peristaltic pump connected to the reservoir;
an ozone generator configured to generate ozone;
a nebulizer configured to convert disinfectant into a vapor, wherein the peristaltic pump is configured to deliver disinfectant from the reservoir to the nebulizer;
a fan configured to circulate air, including ozone, through the system and chamber;
an inlet configured to allow air to flow into the system; and
an outlet configured to allow air to flow out of the system.

27. The system of claim 26, wherein the inlet is fluidically connected to the ozone generator.

28. The system of claim 27, further comprising a valve that is configured to be opened or closed to allow or prevent air flow from the inlet to the ozone generator.

29. The system of claim 26, further comprising a valve that is configured to control fluid flow between the fan and the ozone generator.

30. The system of claim 27, wherein the valve is configured to close such that fluid flow from the fan is blown through the outlet.

31. The system of claim 26, wherein the disinfectant concentration is between about 30% to 60%.

32. The system of claim 26, wherein the disinfectant concentration is about 50%.

33. The system of claim 26, wherein the disinfectant is hydrogen peroxide.

34. The system of claim 31, wherein the hydrogen peroxide concentration is about 50%.

35. The system of claim 26, wherein the reservoir comprises a replaceable cartridge.

36. The system of claim 26, wherein the system is configured to operate at a temperature between 20° C. to 25° C.

37. The system of claim 26, wherein the peristaltic pump is configured to provide a flow rate of less than about 1 ml/min of hydrogen peroxide.

38. The system of claim 26, wherein the system is configured to operate with a relative humidity between 20% and 60%.

39. The system of claim 26, wherein the inlet comprises a high efficiency particulate air (HEPA) filter.

40. The system of claim 26, wherein the outlet comprises an activated carbon filter or a high efficiency particulate air (HEPA) filter.

41. The system of claim 26, further including a sensor disposed in the chamber and configured to sense a level of at least one of humidity, pressure, and temperature within the chamber.

42. The system of claim 36, wherein the peristaltic pump is configured to receive the cartridge.

43. The system of claim 42, wherein the peristaltic pump is configured to deliver a predetermined quantity of hydrogen peroxide solution to the nebulizer.

44. The system of claim 36, wherein the cartridge can include the disinfectant.

45. A method for reducing viable microbial burden on a surface, the method comprising:
placing at least one item into a chamber configured to contain the at least one item;
activating a conditioning phase, the conditioning phase comprising:
activating a fan to circulate air in a closed loop to circulate the chamber,
activating an ozone generator to generate ozone,
activating the fan to circulate air, including the ozone, in the closed loop between the ozone generator and the chamber;
activating a disinfection phase, the disinfection phase comprising:
pumping disinfectant with a peristaltic pump from a reservoir to a nebulizer,
converting disinfectant into a vapor with the nebulizer,
activating the fan to circulate air, including the vapor, in the closed loop between the nebulizer and the chamber, and
activating the fan to circulate air, including the ozone, in the closed loop between the ozone generator and the chamber;
activating a post-disinfection conditioning phase, the post-disinfection conditioning phase comprising:
activating an ozone generator to generate ozone, and
activating the fan to circulate air, including the ozone, in the closed loop between the ozone generator, the nebulizer, and the chamber,
activating a system clearing phase, the system clearing phase comprising:
activating a valve to allow air to flow into the system through an inlet,
activating a valve to allow air to flow out of the system through an outlet, and
activating the fan to introduce the air through the inlet, into the chamber, and exhaust through the outlet.

46. The method of claim 45, wherein the method is performed in about 10 minutes.

47. The method of claim 45, wherein the conditioning phase is about 150 seconds in duration.

48. The method of claim 45, wherein the post-disinfection conditioning phase is about 2 minutes in duration.

49. The method of claim 45, wherein the sterilization or disinfection phase is about 4 minutes and 30 seconds to about 5 minutes in duration.

50. The method of claim 45, wherein the system clearing phase is about 60 seconds.

51. The method of claim 45, wherein the inlet comprises a HEPA filter.

52. The method of claim 45, wherein the system clearing phase further comprises closing a valve to allow the fan to push air through the outlet.

53. The method of claim 45, wherein the outlet comprises an activated carbon filter and a high efficiency particulate air (HEPA) filter.

54. The method of claim 45, further comprising providing the disinfectant at a concentration of between about 30% to 60%.

55. The method of claim 45, further comprising providing the disinfectant at a concentration of about 50%.

56. The method of claim 45, wherein the disinfectant is hydrogen peroxide.

57. The method of claim 56, wherein the hydrogen peroxide is at a concentration of about 50%.

58. The method of claim 45, wherein the reservoir is a replaceable cartridge.

59. The method of claim 45, further comprising performing the method at a temperature between about 20° C. to 25° C.

60. The method of claim 45, further comprising performing the method at a relative humidity between about 20% and 60%.

61. The method of claim 45, further comprising performing the method at an ambient pressure.

62. A automated method for sterilizing or disinfecting at least one item, the method comprising:
receiving at least one item to be sterilized or disinfected into an interior volume of a chamber for sterilization or disinfection, wherein the chamber is part of a system comprising:
an inlet, an outlet port, an ozone generator, a sterilant generator, and a plurality of conduits configured to fluidly connect each of the inlet, sterilant generator, ozone generator, and the chamber;
at least one fan, configured to provide gaseous flow through the system;
a controller; and
a plurality of valves in respective conduits;
activating a conditioning phase by the controller, wherein the conditioning phase is configured to dry a surface of the at least one item in the chamber and internal flow conduits, wherein the controller activates the fan to move air, and wherein the valves are positioned by the controller to provide closed loop flow of air moved by the fan;
activating an disinfection phase by the controller, wherein the exposure phase is configured to disinfect the at least one item, wherein the controller causes the disinfectant generator to begin generating disinfectant, wherein the disinfectant comprises a mist of hydrogen peroxide generated from a solution of hydrogen peroxide in the disinfectant generator at a concentration of about 50%, wherein the valves are positioned by the controller to provide closed loop flow through the nebulizer so that disinfectant is delivered to the chamber for a pre-determined time to disinfect the at least one item;
activating a post-disinfection conditioning phase by the controller, wherein the post-disinfection phase introduces ozone generated by the ozone generator into the chamber containing residual hydrogen peroxide disinfectant to neutralize the disinfectant; and
activating a system clearing phase by the controller, wherein the purge phase includes positioning the valves by the controller to allow open flow and to allow air to be pulled in through the inlet and force the gaseous water vapor and oxygen from the chamber and out the outlet, wherein each of the inlet and outlet comprise a respective filter.

63. The automated method of claim 62, wherein the controller activates the fan to move air through the ozone generator to produce ozone.

64. The automated method of claim 62, wherein the disinfectant comprises a vapor of hydrogen peroxide.

65. The automated method of claim 62, wherein the method operates at a preprogrammed relative humidity between about 20% to 60%.

66. The automated method of claim 62, wherein the conditioning phase is activated for about 180 seconds.

67. The automated method of claim 62, wherein the disinfection phase is activated for about 4 minutes and 30 seconds.

68. The automated method of claim 62, wherein the post-disinfection conditioning phase is activated for about 120 seconds.

69. The automated method of claim 62, wherein the system clearing phase is activated for about 60 seconds.

70. The automated method of claim 62, wherein the system is configured to receive a cartridge.

71. The automated method of claim 26, wherein the method operates between an ambient temperature between about 20° C. to 25° C.

72. The automated method of claim 26, wherein the sterilant is delivered by a peristaltic pump.

73. The automated method of claim 26, wherein at least one of the filters of the inlet and outlet is a HEPA filter.

74. The automated method of claim 26, wherein at least one of the filters of the inlet and outlet is a charcoal filter.
